(19) United States
(12) Patent Application Publication
(10) Pub. No.: US 2010/0136607 A1
(43) Pub. Date: Jun. 3, 2010

(54) SENSOR DEVICE

(75) Inventors: Girish Chandra Upreti, Hillcrest Hamilton (NZ); Yanming Wang, Hillcrest Hamilton (NZ); Alona Swee Kueh, Hamilton (NZ)

Correspondence Address: MERCHANT & GOULD PC, P.O. BOX 2903, MINNEAPOLIS, MN, 55402-0903, US

(73) Assignee: THE NEW ZEALAND INSTITUTE FOR PLANT AND FOOD RESEA, Auckland (NZ)

(21) Appl. No.: 12/520,727

(22) PCT Filed: Dec. 20, 2007
(86) PCT No.: PCT/NZ2007/000376

§ 371 Date: Jan. 18, 2010

Related U.S. Application Data
(60) Provisional application No. 60/876,523, filed on Dec. 22, 2006.

(51) Int. Cl. C12Q 1/04; C12M 1/34; G01N 33/02; G01N 33/497; G01N 21/80

(52) U.S. Cl. 435/34; 435/287.1; 436/133; 436/20; 422/55; 422/57

(57) ABSTRACT

The invention relates to a sensor device for detecting CO₂ wherein the said device has (a) a support base, (b) an acid-base indicator composition that stably changes colour upon exposure to CO₂; and (c) a viewer cover, and wherein least (a) and (c) is CO₂ permeable and wherein (b) includes at least one acid-base indicator that has a pKₗₙ of or above 9.5 and (b) is disposed between (a) and (c) such that the colour change of (b) can be observed through (c). The sensor device of the present invention may be utilized to monitor the ripening process of fresh produces such as fruits, the fermentation or spoilage of food, and/or activity of micro-organisms such as soil and root nodule bacteria in soil.

### FIELD OF THE INVENTION

The invention relates generally to a sensor device for detecting CO₂. The device has application in the food industry, for example, for monitoring the ripening process of produce or the fermentation or spoilage of food. The device can also be used to monitor the activity of other micro-organisms such as soil and root nodule bacteria.

### BACKGROUND OF THE INVENTION

The emission of CO₂ from produce and other foods can provide valuable information about the state of the produce or food. Produce and other foods undergo many processes that involve emission of CO₂. For example, ripening produce generates CO₂ through the respiration process. Therefore, knowledge of the CO₂ emission of a particular fruit can be used to assess the ripeness of the fruit. Many fruit do not change colour when they ripen. Some fruit may show visible signs of ripening that are difficult for the consumer to interpret. However, measuring CO₂ emissions from fruit provides a convenient, non-invasive means of gauging fruit ripeness.

Microbial action in food during fermentation or food spoilage also generates CO₂. Measuring the CO₂ released from produce and foods allows these processes to be monitored in real-time.

Food spoilage microorganisms such as bacteria and some fungi may be introduced into food by improper handling. Other food spoilage microorganisms are naturally present in the food and multiply as the food ages. Excessive amounts of foods are lost to microbial spoilage even with modern day preservation techniques. In addition, foodbourne illness caused by the transfer of microbial contaminants from food to humans is a big concern in the food industry. In some cases foodbourne illnesses can be life threatening, for example illnesses caused by E. coli 0157:H7.

Food spoilage is not always visible. If the food is enclosed in packaging, spoilage may be difficult to detect. Even if the food is examined directly, it may look and smell fresh while still containing harmful pathogens. To overcome this problem many perishable foods are packaged with an expiry date. The expiry date represents an estimate of the date a food would generally be considered too spoiled for safe consumption. However, this estimate assumes the food will experience average or even ideal conditions such as constant refrigeration. Therefore, the food may be spoiled prior to the expiry date or may still be safe to eat after the expiry date.

To minimise the possibility that food spoils before the expiry date provided, some food manufacturers provide expiry dates well in advance of the likely spoilage date of the food. However, this strategy can have undesirable consequences if experience leads consumers to believe it can be safe to consume food well after its specified expiry date.

The micro-organisms responsible for fermentation also release CO₂. Baker's yeast produces CO₂ by fermentation of dough. CO₂ is also produced in bread making by the action of chemical leaveners such as baking powder and baking soda. Yeast also converts sugar into CO₂ and ethanol in alcoholic fermentation. The dairy industry produces dozens of products from milk and cream that rely on fermentation, for example, cheese, yogurt and the like.

Measuring the emission of CO₂ from fermentation products can be used to monitor the progress and end point of the fermentation process.

Micro-organisms in soil also release CO₂. Organic carbon released from decaying plant matter is oxidised by micro-organisms resulting in CO₂ emissions. Monitoring CO₂ loss from soil provides valuable information about the contribution these processes make to CO₂ accumulation in the atmosphere.

In addition, CO₂ loss from soil may reflect the biological activity of the soil. For example, in pastures of nitrogen-fixing plants such as clover, the amount of CO₂ released from soil provides an indication of the activity of nitrogen-fixing bacteria present in root nodules, and therefore the health of the clover plants or pasture as a whole.

Devices that measure the gaseous emissions of produce and foods are known in the art.

US 2006/0127543 relates to a non-invasive colorimetric ripeness indicator for indicating produce ripeness through an ethylene-related colour change. The role of the fruit hormone ethylene in produce ripening is well known. When produce approaches maturity it releases ethylene. Some of the ethylene is reabsorbed and acts as an autocatalytic stimulant to initiate the production of more ethylene.

The apparatus described in US 2006/0127543 uses a colorimetric redox indicator which changes colour in response to ethylene absorption. However, not all produce or food release ethylene and therefore the apparatus is limited in application.

PCT publication WO 2006/062870 describes a food freshness sensor that uses a solution that changes from green to orange in response to a 0.5% concentration of an acidic gas such as CO₂ The solution is packaged in a gas permeable container that allows diffusion of the CO₂.

However, the colour change in the sensor is reversible. CO₂ diffusion out of the sensor may cause the colour to revert back towards the original (control) colour. Therefore, under certain conditions the sensor may indicate the food to be fresh when it is not.

U.S. Pat. No. 6,589,761 relates to a device and method for detecting bacteria in food comprising a CO₂ impermeable transparent support base, a CO₂ permeable cover and an indicator that changes colour in response to a decrease in pH. The specification lists a great number of indicators as suitable for use in the invention but it is unlikely that many of the indicators would change colour in response to CO₂ produced by food bacteria. In addition, the device does not provide a stable colour change when exposed to CO₂ and therefore may indicate a lower CO₂ exposure than has occurred.

Accordingly, there is a need for a device that provides a stable colour change in response to CO₂ and that can be tuned for different applications in the food industry or which at least provides the public with a useful choice.

### SUMMARY OF THE INVENTION

The invention relates generally to a device for detecting CO₂. The device comprises a colorimetric sensor in the form of an acid-base indicator composition that stably changes colour in response to an increasing CO₂ concentration.

In one aspect the invention provides a device for detecting CO₂ comprising:
(a) a support base;
(b) an acid-base indicator composition that stably changes colour upon exposure to CO₂, and
(c) a viewing cover;
   wherein at least one of (a) and (c) is CO₂ permeable and wherein (b) includes at least one acid-base indicator that has a pKₗₙ of or above about 9.5 and (b) is disposed between (a) and (c) such that the colour change of (b) can be observed through (c).

In one embodiment the at least one acid-base indicator has a pKₗₙ of about 10.3 or above. Preferably, the at least one acid-base indicator has a pKₗₙ of about 10.6 or above, more preferably a pKₗₙ of about 11.0 or above.

In one embodiment the at least one acid-base indicator has a pKₗₙ of about 10.3 to about 12. Preferably, the at least one acid-base indicator has a pKₗₙ of about 10.6 to about 12, more preferably a pKₗₙ of about 11 to about 12.

In one embodiment the pH of the acid-base indicator composition is greater than about 10.5. Preferably, the pH of the acid-base indicator composition is greater than about 11, more preferably, greater than about 11.5 and most preferably, greater than about 12.

In one embodiment the pH of the acid-base indicator composition is between about 10.5 and about 13.5. Preferably, the pH of the acid-base indicator composition is between about 11 and about 13.5, more preferably, between about 11.5 and about 13.5 and most preferably, between about 12 and about 13.5.

In one embodiment the pH of the acid-base indicator composition is greater than the pKᵢₙ of the acid-base indicator.

In one embodiment the acid-base indicator composition comprises at least one buffer with at least one pKₐ of about 10 or above. Preferably the acid-base indicator composition comprises at least one buffer with at least one pKₐ of about 10.3 or above.

In one aspect the invention provides a device for detecting CO₂ comprising:
(a) a support base;
(b) an acid-base indicator composition, an
(c) a viewing cover;
   wherein at least one of (a) and (c) is CO₂ permeable and wherein (b) includes at least one acid-base indicator that has a pKₗₙ of or above about 9.5 and (b) is disposed between (a) and (c) such that (b) can be observed through (c).

In one embodiment the at least one acid-base indicator has a pKₗₙ of about 10.3 or above. Preferably, the at least one acid-base indicator has a pKₗₙ of about 10.6 or above, more preferably a pKᵢₙ of about 11.0 or above.

In one embodiment the at least one acid-base indicator has a pKₗₙ of about 10.3 to about 12. Preferably, the at least one acid-base indicator has a pKₗₙ of about 10.6 to about 12, more preferably a pKₗₙ of about 11 to about 12.

In one embodiment the pH of the acid-base indicator composition is greater than about 10.5. Preferably, the pH of the acid-base indicator composition is greater than about 11, more preferably, greater than about 11.5 and most preferably, greater than about 12.

In one embodiment the pH of the acid-base indicator composition is between about 10.5 and about 13.5. Preferably, the pH of the acid-base indicator composition is between about 11 and about 13.5, more preferably, between about 11.5 and about 13.5 and most preferably, between about 12 and about 13.5.

In one embodiment the pH of the acid-base indicator composition is greater than the pKₗₙ of the acid-base indicator.

In one embodiment the acid-base indicator composition comprises at least one buffer with at least one pKₐ of about 10 or above. Preferably the acid-base indicator composition comprises at least one buffer with at least one pKₐ of about 10.3 or above.

In one aspect the invention provides a device for detecting CO₂ comprising:
(a) a support base;
(b) an acid-base indicator composition of pH greater than about 10 that stably changes colour upon exposure to CO₂, and
(c) a viewing cover;
   wherein at least one of (a) and (c) is CO₂ permeable and wherein (b) includes at least one acid-base indicator that has a pKₗₙ of or above about 9.5 and (b) is disposed between (a) and (c) such that the colour change of (b) can be observed through (c).

In one embodiment the at least one acid-base indicator has a pKₗₙ of about 10.3 or above. Preferably, the at least one acid-base indicator has a pKₗₙ of about 10.6 or above, more preferably a pKₗₙ of about 11.0 or above.

In one embodiment the at least one acid-base indicator has a pKₗₙ of about 10.3 to about 12. Preferably, the at least one acid-base indicator has a pKₗₙ of about 10.6 to about 12, more preferably a pKₗₙ of about 11 to about 12.

In one embodiment the pH of the acid-base indicator composition is greater than about 10.5. Preferably, the pH of the acid-base indicator composition is greater than about 11, more preferably, greater than about 12.

In one embodiment the pH of the acid-base indicator composition is between about 10.5 and about 13.5. Preferably, the pH of the acid-base indicator composition is between about 11 and about 13.5, more preferably, between about 11.5 and about 13.5 and most preferably, between about 12 and about 13.5.

In one embodiment the pH of the acid-base indicator composition is greater than the pKₗₙ of the acid-base indicator.

In one embodiment the acid-base indicator composition comprises at least one buffer with at least one pKₐ of about 10 or above. Preferably the acid-base indicator composition comprises at least one buffer with at least one pKₐ of about 10.3 or above.

In one aspect the invention provides a device for detecting CO₂ emitted by food micro-organisms comprising:
(a) a support base;
(b) an acid-base indicator composition that stably changes colour upon exposure to CO₂, and
(c) an at least partially transparent cover;
   wherein at least one of (a) and (c) is CO₂ permeable and wherein (b) includes at least one acid-base indicator that has a pKₗₙ of or above about 9.5 and (b) disposed between (a) and (c) such that the colour change of (b) can be observed through (c).

In one embodiment the at least one acid-base indicator has a pKₗₙ of about 10.3 or above. Preferably, the at least one acid-base indicator has a pKₗₙ of about 10.6 or above, more preferably a pKₗₙ of about 11.0 or above.

In one embodiment the at least one acid-base indicator has a pKₗₙ of about 10.3 to about 12. Preferably, the at least one acid-base indicator has a pKₗₙ of about 10.6 to about 12, more preferably a pKₗₙ of about 11 to about 12.

In one embodiment the pH of the acid-base indicator composition is greater than about 10.5. Preferably, the pH of the acid-base indicator composition is greater than about 11, more preferably, greater than about 12.

In one embodiment the pH of the acid-base indicator composition is between about 10.5 and about 13.5. Preferably, the pH of the acid-base indicator composition is between about 11 and about 13.5, more preferably, between about 11.5 and about 13.5 and most preferably, between about 12 and about 13.5.

In one embodiment the pH of the acid-base indicator composition is greater than the pKₗₙ of the acid-base indicator.

In one embodiment the acid-base indicator composition comprises at least one buffer with at least one pKₐ of about 10 or above. Preferably the acid-base indicator composition comprises at least one buffer with at least one pKₐ of about 10.3 or above.

In one embodiment the food micro-organisms are food spoilage micro-organisms.

In other embodiment the food micro-organisms are fermentation micro-organisms.

In one aspect the invention provides a device for detecting CO₂ emitted by soil micro-organisms comprising:
(a) a support base;
(b) an acid-base indicator composition that stably changes colour upon exposure to CO₂, and
(c) a viewing cover;
   wherein at least one of (a) and (c) is CO₂ permeable and wherein (b) includes at least one acid-base indicator that has a pKₗₙ of or above about 9.5 and (b) disposed between (a) and (c) such that the colour change of (b) can be observed through (c).

In one embodiment the at least one acid-base indicator has a pKₗₙ of about 10.3 or above. Preferably, the at least one acid-base indicator has a pKₗₙ of about 10.6 or above, more preferably a pKₗₙ of about 11.0 or above.

In one embodiment the at least one acid-base indicator has a pKₗₙ of about 10.3 to about 12. Preferably, the at least one acid-base indicator has a pKₗₙ of about 10.6 to about 12, more preferably a pKₗₙ of about 11 to about 12.

In one embodiment the pH of the acid-base indicator composition is greater than about 10.5. Preferably, the pH of the acid-base indicator composition is greater than about 11, more preferably, greater than about 12.

In one embodiment the pH of the acid-base indicator composition is between about 10.5 and about 13.5. Preferably, the pH of the acid-base indicator composition is between about 11 and about 13.5, more preferably, between about 11.5 and about 13.5 and most preferably, between about 12 and about 13.5.

In one embodiment the pH of the acid-base indicator composition is greater than the pKₗₙ of the acid-base indicator.

In one embodiment the acid-base indicator composition comprises at least one buffer with at least one pKₐ of about 10 or above. Preferably the acid-base indicator composition comprises at least one buffer with at least one pKₐ of about 10.3 or above.

In one embodiment the soil micro-organisms are nitrogen-fixing bacteria present in root nodules of clover and other legumes.

For the devices of the invention described above:

In one embodiment the at least one acid-base indicator is selected from the group comprising Alizarin yellow R, Alizarin yellow GG, Alizarin CI58000, Thiazole yellow G, thymophthalein, phenolphthalein, phenolthymolphthalein, brilliant orange, Tropaeolin 000, Tropaeolin 00, Tropaeolin 0, and mixtures thereof.

In one embodiment the at least one acid-base indicator is selected from the group comprising Alizarin yellow R, Alizarin CI58000, Alizarin yellow GG and Thiazole yellow G.

Preferably, the at least one acid-base indicator is Alizarin Yellow R.

In one embodiment the concentration of the at least one acid-base indicator in the acid-base indicator composition is between about 0.5 mM to about 10 mM. Preferably, the concentration of the at least one acid-base indicator in the acid-base indicator composition is between about 1 mM to about 8 mM, more preferably, between about 2 mM to about 6 mM.

In one embodiment the acid-base indicator composition comprises an alkali. Preferably, the alkali is selected from the group comprising one or more metal hydroxides such as NaOH, KOH, LiOH, Ca(OH)₂, Mg(OH)₂, Ba(OH)₂.

More preferably the alkali is aqueous NaOH or aqueous KOH.

In one embodiment the concentration of the alkali in the acid-base indicator composition is between about 50 to about 200 mM. Preferably, the concentration of the alkali is between about 75 to about 175 mM, more preferably, between about 80 to about 150 mM.

In another embodiment the concentration of the alkali in the acid-base indicator composition is between about 35 to about 200 mM. Preferably, the concentration of the alkali is between about 45 to about 175 mM, more preferably, between about 45 to about 140 mM.

In one embodiment the acid-base indicator composition comprises a buffer system. Preferably the buffer system comprises at least one buffer with at least one pKₐ of about 10 or above, more preferably, 10.3 or above. Preferably, the buffer system comprises at least one buffer selected from the group comprising phosphate buffers, CAPS, tricine, glycine, CAPSO, taurine and 2-(N-cyclohexylamino)ethanesulfonic acid (CHES).

Preferably the phosphate buffer is disodium hydrogen phosphate dihydrate (Na₂HPO₄.2H₂O) or a salt, such as a potassium salt of phosphoric acid.

In one embodiment the buffer system includes one or more buffers that in combination have at least two pKₐs that between them span the pH range of the acid-base indicator colour change. Preferably, the buffer system comprises disodium hydrogen phosphate dihydrate and CAPS.

In one embodiment the concentration of the at least one or more buffers in the acid-base indicator composition is between about 5 mM to about 40 mM, preferably between about 10 mM to about 30 mM and more preferably, between about 10 mM to about 20 mM.

In one embodiment the acid-base indicator composition further comprises a salt such as NaCl or KCl. In one embodiment the concentration of the salt in the acid-base indicator composition is between about 20 to about 300 mM. Preferably, the concentration of the salt is between about 50 to about 200 mM, more preferably, between about 75 and 150 mM.

In one embodiment the acid-base indicator composition comprises NaCl or KCl, preferably NaCl.

In one embodiment the acid-base indicator composition further comprises a chelating agent. Preferably the chelating agent is ethylenediaminetetraacetic acid (EDTA) or ethylene glycol-bis(2-aminoethylether)-N,N,N⁻,N⁻-tetraacetic acid (EGTA). Preferably the chelating agent is EDTA.

In one embodiment the acid-base indicator composition further comprises an organic solvent. Preferably, the organic solvent is ethanol.

In one embodiment the acid-base indicator composition comprises a humectant. Preferably, the humectant is glycerol.

In one embodiment the pH of the acid-base indicator composition is greater than about 10.5. Preferably, the pH of the acid-base indicator composition is greater than about 11, more preferably greater than about 11.5 and most preferably, greater than about 12.

In one embodiment the pH of the acid-base indicator composition is between about 10.5 and about 13.5. Preferably, the pH of the acid-base indicator composition is between about 11 and about 13.5, more preferably, between about 11.5 and about 13.5 and most preferably, between about 12 and about 13.5.

In one embodiment the pH of the acid-base indicator composition is greater than the pKₗₙ of the acid-base indicator.

In one embodiment the acid-base composition is disposed within a carrier matrix.

In one embodiment the support base is CO₂ permeable and the viewing cover is CO₂ impermeable.

In another embodiment the support base is CO₂ impermeable and the viewing cover is CO₂ permeable.

In another embodiment both the support base and the viewing cover are CO₂ permeable.

In one embodiment the support base comprises laminate film.

In another embodiment the viewing cover comprises laminate film.

In one embodiment both the support base and the viewing cover comprise laminate film.

In one embodiment the device of the invention may further comprise a colour chart correlating the possible colours of the acid-base indicator composition with the concentration of CO₂. Preferably, the colour chart is integrally formed with the device of the invention.

In one aspect the invention provides a plurality of integrally formed CO₂ detecting devices, each device comprising
(a) a support base;
(b) an acid-base indicator composition that stably changes colour upon exposure to CO₂, and
(c) a viewing cover;
   wherein at least one of (a) and (c) is CO) permeable, and wherein (b) includes at least one acid-base indicator that has a pKₗₙ of or above 9.5 and (b) is disposed between (a) and (c) such that the colour change of (b) can be observed through (c).

In one embodiment the at least one acid-base indicator has a pKₗₙ of about 10.3 or above. Preferably, the at least one acid-base indicator has a pKₗₙ of about 10.6 or above, more preferably a pKₗₙ of about 11.0 or above.

In one embodiment the at least one acid-base indicator has a pKₗₙ of about 10.3 to about 12. Preferably, the at least one acid-base indicator has a pKₗₙ of about 10.6 to about 12, more preferably a pKₗₙ of about 11 to about 12.

In one embodiment the pH of the acid-base indicator composition is greater than about 10.5. Preferably, the pH of the acid-base indicator composition is greater than about 11, more preferably, greater than about 11.5 and most preferably, greater than about 12.

In one embodiment the pH of the acid-base indicator composition is between about 10.5 and about 13.5. Preferably, the pH of the acid-base indicator composition is between about 11 and about 13.5, more preferably, between about 11.5 and about 13.5 and most preferably, between about 12 and about 13.5.

In one embodiment the pH of the acid-base indicator composition is greater than the pKₗₙ of the acid-base indicator.

In one embodiment the acid-base indicator composition comprises at least one buffer with at least one pKₐ of about 10 or above. Preferably the acid-base indicator composition comprises at least one buffer with at least one pKₐ of about 10.3 or above.

In another aspect the invention provides a method for detecting CO₂ from a CO₂ emitting source comprising:
(1) placing on or near the CO, emitting source, a device for detecting CO₂, wherein the device comprises
   (a) a support base;
   (b) an acid-base indicator composition that stably changes colour upon exposure to CO₂, and
   (c) a viewing cover;
   wherein at least one of (a) and (c) is CO₂ permeable and wherein (b) includes at least one acid-base indicator that has a pKₗₙ of or above about 9.5 and (b) is disposed between (a) and (c) such that the colour change of (b) can be observed through (c); and
(2) detecting a colour change in (b).

In one embodiment the device for detecting CO₂ is placed on or up to about 10 cm from the CO₂ emitting source.

In another aspect the invention provides a method for indicating fruit ripeness comprising:

(1) placing on or near the fruit, a device for detecting CO₂ emitted by fruit, wherein the device comprises
(a) a support base;
(b) an acid-base indicator composition that stably changes colour upon exposure to CO₂, and
(c) a viewing cover;
wherein at least one of (a) and (c) is CO₂ permeable and wherein (b) includes at least one acid-base indicator that has a pKₗₙ of or above about 9.5 and (b) is disposed between (a) and (c) such that the colour change of (b) can be observed through (c); and
   (2) detecting a colour change in (b).

Preferably, the device is placed on or up to about 10 cm from the fruit.

The devices of the invention are particularly suited for detecting CO₂ emitted from climacteric fruit. Accordingly, in one embodiment the invention provides a method for indicating fruit ripeness in climacteric fruit.

In another aspect the invention provides a method for indicating food spoilage comprising:

(1) placing on or near food, a device for detecting CO₂ emitted by food spoilage micro-organisms,
wherein the device comprises
(a) a support base;
(b) an acid-base indicator composition that stably changes colour upon exposure to CO₂, and
(c) a viewing cover;
wherein at least one of (a) and (c) is CO₂ permeable and (b) includes at least one acid-base indicator that has a pKₗₙ of or above about 9.5 and (b) is disposed between (a) and (c) such that the colour change of (b) can be observed through (c); and
   (2) detecting a colour change in (b).

Preferably, the device is placed on or up to about 10 cm from the food.

In one embodiment the invention provides a method for indicating food spoilage caused by the group of micro-organisms including but not limited to the group comprising Salmonella enteriitidis, Campylobacter jejuni, Escherichia coli including 0157:H7, Staphylococcus aureus, Shigella dysenteriae, Listeria monocytogenes, clostridium perfringen, Bacillus cereus, Vibrio parahaemolyticus, Vibrio cholerae, Yersinia enterocolitica, Brochrothrix thermosphacta, Pseudomonus sp., Acromonas sp., Lactobacillus plantarum, Lactobacillus fructivorans, Serratia liquefaciens, and Zygosaccharomyces bacilli.

In another aspect the invention provides a method for indicating food fermentation comprising:

(1) placing on or near the food, a device for detecting CO₂ emitted by food fermentation micro-organisms,
wherein the device comprises
(a) a support base;
(b) an acid-base indicator composition that stably changes colour upon exposure to CO₂, and
(c) a viewing cover;
wherein at least one of (a) and (c) is CO₂ permeable and wherein (b) includes at least one acid-base indicator that has a pKₗₙ of or above about 9.5 and (b) is disposed between (a) and (c) such that the colour change of (b) can be observed through (c); and
   (2) detecting a colour change in (b).

Preferably, the device is placed on or up to about 10 cm from the food.

In one embodiment the invention provides a method for indicating food fermentation caused by the group of micro-organisms including but not limited to the group comprising Lactocooccus lactis, Steptococcus thermophilus, Leuconostoc sp., Pediococcus sp., Lactobacillus sp., Bifidobacterium sp., Propiopribacterium sp., Lactobacillus delbruekii, Streptococcus thermophilus, Saccharomyces cerevisiae, Candida rugosa, Kluyveromyces marxianus, Aspergillus oryzae, and the like.

In another aspect the invention provides a method for indicating CO₂ emissions from soil comprising:

(1) placing on or near the soil, a device for detecting CO₂ emitted by soil micro-organisms,
wherein the device comprises
(a) a support base;
(b) an acid-base indicator composition that stably changes colour upon exposure to CO₂, and
(c) a viewing cover;
wherein at least one of (a) and (c) is CO₂ permeable and wherein (b) includes at least one acid-base indicator that has a pKₗₙ of or above about 9.5 and (b) is disposed between (a) and (c) such that the colour change of (b) can be observed through (c); and
   (2) detecting a colour change in (b).

Preferably, the device is placed on or up to about 10 cm from the soil.

In one embodiment the invention provides a method for indicating CO₂ emissions from soil caused by nitrogen-fixing bacteria present in root nodules.

In another aspect the invention provides a kit for indicating fruit ripeness comprising

(1) a device for detecting CO₂ emitted by fruit wherein the device comprises
(a) a support base;
(b), an acid-base indicator composition that stably changes colour upon exposure to CO₂, and
(c) a viewing cover;
wherein at least one of (a) and (c) is CO₂ permeable and wherein (b) includes at least one acid-base indicator that has a pKₗₙ of or above 9.5 and (b) is disposed between (a) and (c) such that the colour change of (b) can be observed through (c); and
   (2) a colour chart correlating the possible colours of (b) with the ripeness of the fruit.

In another aspect the invention provides a kit for indicating food spoilage comprising

(1) a device for detecting CO₂ emitted by food spoilage micro-organisms wherein the device comprises
(a) a support base;
(b) an acid-base indicator composition that stably changes colour upon exposure to CO₂, and
(c) a viewing cover;
wherein at least one of (a) and (c) is CO₂ permeable and wherein (b) includes at least one acid-base indicator that has a pKₗₙ of or above 9.5 and (b) is disposed between (a) and (c) such that the colour change of (b) can be observed through (c); and
   (2) a colour chart correlating the possible colours of (b) with the degree of spoilage of the food.

In another aspect the invention provides a kit for indicating food fermentation comprising

(1) a device for detecting CO₂ emitted by food fermentation micro-organisms wherein the device comprises
(a) a support base;
(b) an acid-base indicator composition that stably changes colour upon exposure to CO₂, and
(c) a viewing cover;
wherein at least one of (a) and (c) is CO₂ permeable and wherein (b) includes at least one acid-base indicator that has a pKₗₙ of or above 9.5 and (b) is disposed between (a) and (c) such that the colour change of (b) can be observed through (c); and
   (2) a colour chart correlating the possible colours of (b) with the degree of fermentation of the food.

In another aspect the invention provides a kit for indicating CO₂ emissions from soil comprising

(1) a device for detecting CO₂ emitted by soil micro-organisms wherein the device comprises
(a) a support base;
(b) an acid-base indicator composition that stably changes colour upon exposure to CO₂, and
(c) a viewing cover;
wherein at least one of (a) and (c) is CO₂ permeable and wherein (b) includes at least one acid-base indicator that has a pKₗₙ of or above 9.5 and (b) is disposed between (a) and (c) such that the colour change of (b) can be observed through (c); and
   (2) a colour chart correlating the possible colours of (b) with the levels of CO₂ emissions from the soil.

For the methods and kits outlined above:

In one embodiment the at least one acid-base indicator has a pKₗₙ of about 10.3 or above. Preferably, the at least one acid-base indicator has a pKₗₙ of about 10.6 or above, more preferably a pKₗₙ of about 11.0 or above.

In one embodiment the at least one acid-base indicator has a pKₗₙ of about 10.3 to about 12. Preferably, the at least one acid-base indicator has a pKₗₙ of about 10.6 to about 12, more preferably a pKₗₙ of about 11 to about 12.

In one embodiment the pH of the acid-base indicator composition is greater than about 10.5. Preferably, the pH of the acid-base indicator composition is greater than about 11, more preferably, greater than about 11.5, most preferably, greater than about 12.

In one embodiment the pH of the acid-base indicator composition is between about 10.5 and about 13.5. Preferably, the pH of the acid-base indicator composition is between about 11 and about 13.5, more preferably, between about 11.5 and about 13.5 and most preferably, between about 12 and about 13.5.

In one embodiment the pH of the acid-base indicator composition is greater than the pKₗₙ of the acid-base indicator.

In one embodiment the acid-base indicator composition comprises at least one buffer with at least one pKₐ of about 10 or above. Preferably the acid-base indicator composition comprises at least one buffer with at least one pKₐ of about 10.3 or above.

The invention may also broadly be said to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE DRAWINGS

FIG. 1A shows a schematic view of a preferred embodiment of the sensor device of the invention.

FIG. 1B shows a plan view of a preferred embodiment of the sensor device of the invention.

FIG. 1C shows a plan view of a plurality of sensor devices of the invention.

FIG. 1D shows a plan view of the sensor device of the invention integrally formed with a reference colour chart

FIG. 2 is a graph showing the colour response of three sensor devices of the invention when incubated at room temperature in the presence of 1% CO₂.

FIG. 3 is a photograph of a preferred embodiment of the sensor device of the invention after exposure to different concentrations of CO₂.

FIG. 4 is a graph showing the colour response of nine sensor devices of the invention when incubated at room temperature in the presence of 1% CO₂.

FIG. 5 is a photograph showing the colour response of three sensor devices of the invention (HRC) and other sensor devices (FFS) after 44 hours in 0%, 0.5%, 1.0% and 2.0% CO₂. A, B and C represent sensors of increasing sensitivity for both the HRC and FFS sensors. All of the sensors used laminate film type A 1.

FIG. 6 is a photograph showing the colour response of the sensor devices incubated for 44 hours at 2% CO₂, as shown in FIG. 5, following exposure to air for 24 hours.

FIG. 7 is a graph showing the colour response of three sensor devices of the invention when exposed to yeast fermentation of flour dough for 20 hours. The graph also shows the CO₂ concentration increasing as yeast fermentation progresses.

FIGS. 8A and AB are photographs showing the colour response of three sensor devices of the invention when exposed to yeast fermentation of flour dough for 6.5 hours. FIG. 8A shows the colour of the sensors at 0 hours, FIG. 8B at 6.5 hours. The labels A, 55 and B correspond to sensors T-045, T-055 and T-088, respectively.

FIG. 9 is a graph showing the colour response of three sensor devices of the invention when exposed to CO₂ released from the soil. The graph also shows CO₂ concentration increasing as CO₂ is emitted from the soil. Autoclaved soil provides a control.

FIG. 10A-10D are photographs showing the colour response of three sensor devices of the invention when incubated with a soil sample over 144 hours. FIG. 10A shows the sensors at 0 hours, 10B at 48 hours, 10C at 96 hours and 10D at 144 hours. A, B and C refer to the sensitivity of the devices; A=T-045, B=1-088 and C=T-140. Autoclaved soil provided a control.

FIGS. 11A-11E are photographs showing the colour response of four sensor devices of the invention during incubation in 1% CO₂ for 187 hours followed by exposure to air for 25 hours. FIG. 11A shows the colour of the sensors at 0 hours of (from left to right) Alizarin Yellow R, Alizarin CI58000, Alizarin GG and Thiazol Yellow G. FIGS. 12B-12E show the sensors at 24, 48 and 187 hours respectively. FIG. 11E shows the sensors 25 hours after the tank has been opened.

FIG. 12 is a photograph showing the colour response of fifteen sensor devices of the invention after exposure to 2% and 10% CO₂ for 4 hours.

FIG. 13 is a photograph showing the colour response of five sensor devices of the invention after exposure to 0%, 1%, 2%, 5%, 7.5% or 10% CO₂ for 28 hours.

FIG. 14 is a graph showing the colour response of three sensor devices of the invention after incubation with kiwifruit for 168 hours. The increase in CO₂ concentration is also shown.

FIGS. 15A-15D are photographs showing the colour response of three sensor devices of the invention with sensitivities T-45, T-88 and T-140 after incubation with kiwifruit for 168 hours. FIGS. 15A-15D show the sensor devices at 0, 72, 144 and 168 hours respectively.

FIGS. 16A-16C are photographs showing how a sensor device of the invention can be incorporated into a milk bottle, to indicate spoilage of the milk. FIG. 16A shows a fresh milk bottle while FIGS. 16B and 16C show the sensor device changing colour as the milk spoils.

### DETAILED DESCRIPTION OF THE INVENTION

### 1. Definitions

The term “acid-base indicator” as used herein means pH indicator and refers to an acid-base pair wherein each member of the pair absorbs light from a different part of the electromagnetic spectrum. In aqueous solution the acid and base forms of the pair are in equilibrium with each other. Changes in the pH of the solution shift the equilibrium in the ratio of the acid and base forms. The pH at which the acid and base pairs of an indicator are at equal concentration is called the pKₗₙ (also referred to as the pKₐ).

The term “acid-base indicator composition” as used herein means a composition comprising at least one acid base indicator. The composition may also contain other reagents. The term “acid-base indicator composition” refers to both (i) a liquid composition placed between the support base and viewing cover of the sensor device of the invention, and (ii) the composition that results when the majority of the solvent has evaporated from the liquid composition of (i). Concentrations and pH values provided refer to those of the liquid composition at the time the composition is placed between the support base and viewing cover of the sensor device.

The term “alkali” as used herein means a water-soluble compound capable of turning litmus blue and reacting with an acid to form a salt and water.

The term “buffer” as used herein means a solution containing either a weak acid and its salt or a weak base and its salt, which is resistant to changes in pH.

The term “CO₂ emitting source” as used herein refers to a product, composition, material or entity that emits CO₂. A CO₂ emitting source may be a living entity such as a micro-organism, plant or animal or a substance containing one or more of these such as food or soil. A CO₂ emitting source may also be a composition such as a carbonated composition or dry ice containing composition. A CO₂ emitting source may also be a device such as a fire extinguisher.

The term “CO₂ permeable” as used herein refers to the property of CO₂ permeability. A CO₂ permeable material allows transmission of CO₂ gas through the material.

The term “comprising” as used herein means “consisting at least in part of”, that is to say when interpreting statements in this specification and claims which include the term, the features, prefaced by that term in each statement, all need to be present but other features can also be present.

The term “food” as used herein means food and beverages and includes but is not limited to meat, poultry, fish, seafood, eggs, dairy products such as milk, cheese, cream, cottage cheese and yogurt, grains, flour and cereal products, vegetables, fruit, legumes, rice, wine and beer.

The term “food fermentation” as used herein means the process by which complex organic molecules are broken down into smaller molecules by fermentation micro-organisms. Food fermentation includes alcoholic fermentation and lactic acid fermentation.

The term “food micro-organism” as used herein means a bacteria, virus, yeast or mould present in food either endogenously or by addition. Food micro-organisms include food spoilage micro-organisms. These micro-organisms may cause foodbourne illness if the food is consumed or may merely spoil the food. Food micro-organisms also include fermentation micro-organisms.

The term “food spoilage” as used herein means the process of decay or deterioration of food colour, flavour, odour or consistency of a food product.

The term “soil micro-organism” as used herein means a micro-organism that lives in the soil and produces CO₂.

The term “stably changes colour” as used herein means that the colour change in response to CO₂ exposure is substantially irreversible. Following a colour change from an original (control colour) to a colour that correlates with a particular CO₂ exposure, the colour will remain substantially the same and will not noticeably change back towards its original colour when the CO₂ source is removed. For example, an acid-base indicator composition containing an acid-base indicator capable of changing colour from red to yellow through orange and vice versa will stably change colour if the colour change from red to yellow in response to CO₂ exposure is substantially irreversible.

The term “support base” as used herein means a base onto which the acid-base indicator composition can be placed. The support base can be transparent or opaque, and can be made of any suitable material known in the art.

The term “viewing cover” as used herein means a cover that is transparent or sufficiently transparent in at least some portion thereof such that the colour of any material behind the cover can be observed.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### 2. Sensor Devices of the Invention

The invention relates generally to a sensor device for detecting CO₂. The device comprises a colorimetric sensor that stably changes colour in response to an increasing CO₂ concentration.

In one aspect the invention provides a device for detecting CO₂ comprising:
(a) a support base;
(b) an acid-base indicator composition that stably changes colour upon exposure to CO₂, and
(c) a viewing cover;
   wherein at least one of (a) and (c) is CO₂ permeable, and wherein (b) includes at least one acid base indicator that has a pKₗₙ of above about 9.5 and (b) is disposed between (a) and (c) such that the colour change of (b) can be observed through (c).

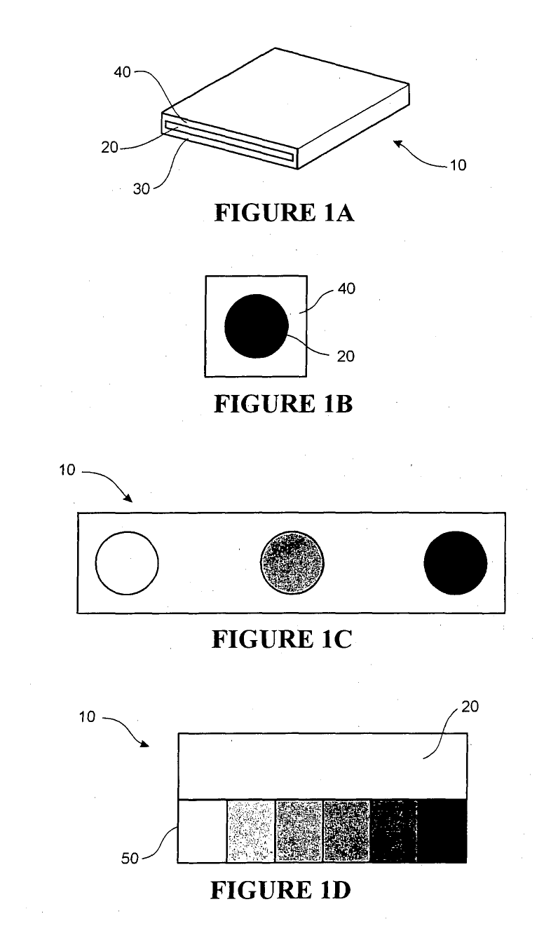

Preferred embodiments of the invention are shown in FIG. 1.

As shown in FIG. 1A the sensor device of the invention (10) comprises an acid-base indicator composition (20) disposed between a support base (30) and a viewing cover (40).

As shown in FIG. 1B the colour change of the acid-base indicator composition (20) in response to CO₂ can be observed through the viewing cover (40).

FIG. 1C shows a plurality of sensor devices of the invention (10) each tuned to change colour at a different CO₂ concentration.

As shown in FIG. 1D, the sensor devices of the invention (10) may also incorporate a reference colour chart (50). The colour change of the acid-base indicator composition (20) in response to an increasing CO₂ concentration can be compared to the reference colour shown in the reference colour chart (50).

The key to the sensor device of the invention is the acid-base indicator composition that is disposed between the support base and the viewing cover.

### 3. The Acid-Base Indicator Composition

The acid-base indicator composition changes colour upon reaction with CO₂. This colour change is substantially irreversible so the colour will remain the same even if the CO₂ is no longer present in the composition.

In an aqueous solution at pH greater than about 6.0, CO₂ dissolves in the water to form an equilibrium with carbonic acid. Carbonic acid reversibly dissociates into hydrogen ions and bicarbonate and carbonate ions as shown below.

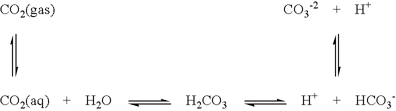

When an acid-base indicator is present in the solution the hydrogen ions generated by the above reaction decrease the pH of the solution. This pH change shifts the equilibrium existing between the acid-base indicator pair. At higher pH the acid-base indicator is deprotonated, or in its basic form. At lower pH, the acid-base indicator is protonated, or in its acid form.

Therefore, a pH change in an acid-base indicator solution causes the solution to change colour. For each change in pH unit, the colour change observed will depend on the colours of each acid-base pair and the concentration of acid-base indicator present.

Generally, the colour change associated with acid-base indicators is reversible. For example, when the CO₂ concentration reduces due to diffusion of the gas out of solution, the pH will increase and the acid-base indicator will revert to its acid form, causing the colour to change.

The present invention provides a sensor device for detecting CO₂ that displays a stable colour change in response to CO₂. Once the device has indicated a certain level of CO₂, it will not revert back to its original colour should the CO₂ concentration subsequently decrease.

Without being bound by theory, it is believed that the stable colour change results from the choice of acid-base indicator used, in conjunction with the other components of the acid-base indicator composition.

The dissolution of CO₂ in water forms a bicarbonate-carbonate buffer system with two pKₐ values, 6.35 and 10.33. The acid-base indicator composition used in the sensor devices of the invention has a high pH. For the sensor devices made using Alizarin yellow R, the pH of the acid-base indicator composition is about 13. At this pH, the acid-base indicator is in its deprotonated form (red for Alizarin yellow R).

As CO₂ diffuses into the device and dissolves in the acid-base indicator composition, hydrogen ions are released and the pH lowers, shifting the equilibrium between the protonated and unprotonated forms of the acid-base indicator. When the acid-base indicator composition has absorbed sufficient CO₂, the acid-base indicator will be mainly in its protonated form (yellow for Alizarin yellow R). If CO₂ diffuses out of the sensor device the pH should rise. However, the buffering action of the bicarbonate-carbonate system, in conjunction with the buffer added to the acid-base indicator composition, ensures that the pH cannot rise higher than 10.3.

This pH is not high enough to significantly change the ratio of deprotonated to protonated forms of Alizarin yellow R, therefore the colour does not change. Consequently, the colour change is stable and does not revert to the original “low CO₂” colour, even when CO₂ is removed from the system.

Similar considerations apply when the sensor devices of the invention are made using other acid-base indicators or combinations of acid-base indicators. The starting pH of the acid-base composition, the buffer system and the acid-base indicator should be selected to ensure that once exposure to CO₂ has effected a colour change, removal of the CO₂ from the acid-base indicator composition cannot change the pH sufficiently to change the colour of the acid by indicator composition.

Acid-base indicators with high pKₗₙ values are suitable for use in the devices of the invention. In one embodiment the acid-base indicator composition comprises at least one acid-base indicator with a pKₗₙ of or about 9.5. Preferably, the at least one acid-base indicator has a pKₗₙ of between about 10 and about 12, more preferably, a pKₗₙ of about 11.

In one embodiment the at least one acid-base indicator has a pKₗₙ of about 10.3 to about 12. Preferably, the at least one acid-base indicator has a pKₗₙ of about 10.6 to about 12, more preferably a pKₗₙ of about 11 to about 12.

In one embodiment the at least one acid-base indicator has a pKₗₙ of about 10.3 or above.

In one embodiment the at least one acid-base indicator is selected from the group comprising Alizarin yellow R, Alizarin CI58000, Alizarin yellow GG, Thiazole yellow G, thymophthalein, phenolthymolphthalein, brilliant orange, Tropaeolin 000, Tropaeolin 00, Tropaeolin 0, and mixtures thereof.

In one embodiment the at least one acid-base indicator is selected from the group comprising Alizarin yellow R, Alizarin CI58000, Alizarin yellow GG and Thiazole yellow G.

Preferably the at least one acid-base indicator is Alizarin yellow R.

In one embodiment the acid-base indicator composition of the invention is made by combining a dye solution with a reagent solution. The dye solution comprises the acid-base indicator dissolved in an appropriate solvent and optionally a humectant. The reagent solution comprises the remaining components including the alkali and the buffer.

The dye solution is combined with the reagent solution to give the acid-base indicator composition for use in the invention. However, it will be understood that the acid-base indicator composition can be prepared by any method.

An example of the preparation of an acid-base indicator composition is provided in Example 1.

The concentration of the at least one acid-base indicator in the acid-base indicator composition depends on the nature of the acid-base indicator. The concentration must be sufficiently high that a clear colour change can be observed in response to exposure to CO₂. However, the concentration must be low enough for the acid-base indicator to remain in solution in the composition.

The optimal concentration will depend on the nature of the acid-base indicator.

In one embodiment the concentration of the at least one acid-base indicator in the acid-base indicator composition is between about 0.1 mM to about 10 mM. Preferably, the concentration of the at least one acid-base indicator in the acid-base indicator composition is between about 1 mM to about 8 mM, more preferably, between about 2 mM to about 6 mM.

In one embodiment the acid-base indicator is Alizarin yellow R at a concentration of between about 2 to 6 mM, preferably about 5 mM.

In one embodiment the acid-base indicator composition stably changes from red to yellow upon exposure to CO₂. However, the stable colour change may be a change from any colour including colourless, to any other colour, including colourless, provided that there is sufficient distinction between the two colour states to be detected by the naked eye.

Example 8 describes the production of sensors of the invention using four different acid-base indicators. The devices were exposed to 1% CO₂ for 187 hours.

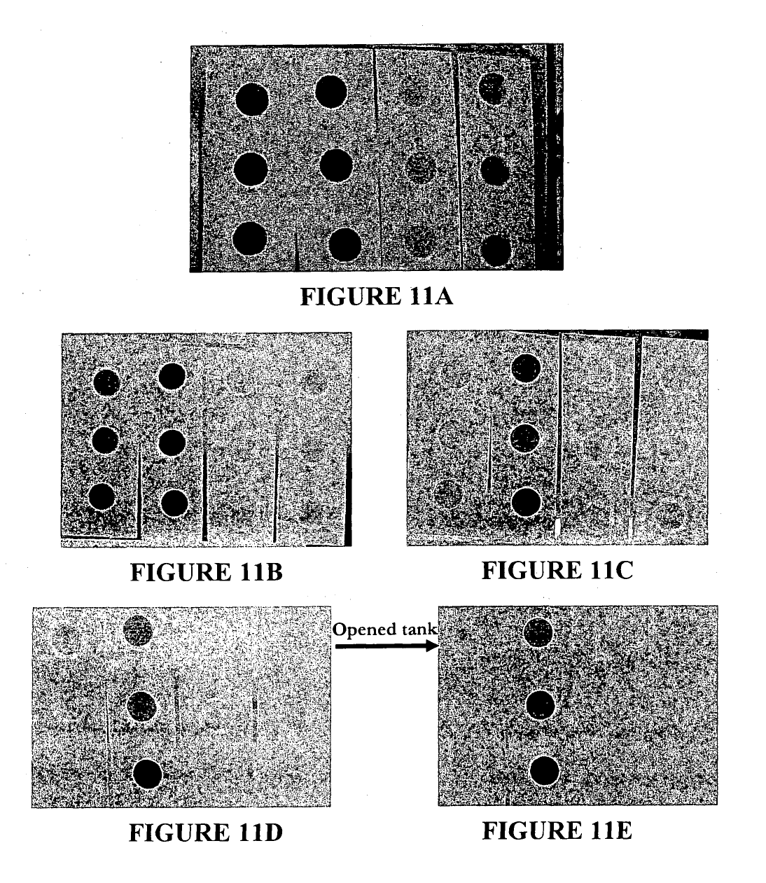

The sensor devices using the acid-base indicator Alizarin CI58000 changed from blue to purple. Those using Alizarin Yellow GG changed from bright yellow/orange to pale yellow and those using Thiazol Yellow G changed from orange to yellow. The results can be seen in FIG. 11.

In one embodiment the acid-base indicator composition comprises an alkali. Preferably the alkali is selected from the group comprising one or more metal hydroxides such as NaOH, KOH, LiOH, Ca(OH)₂, Mg(OH)₂, Ba(OH)₂.

Other organic bases may also be used such as 2-amino-2-methyl-1-propanol (AMP); 2-amino-2-methyl-1,3-propandiol (AMPD); N-(1,1-dimethyl-2-hydroxyethyl)-3-amino-2-hydroxypropansulfonic acid (AMPSO); 3-(cyclohexylamino)-1-propanesulfonic acid (CAPS); and 3-(cyclohexylamino)-2-hydroxy-1-propane sulfonic acid (CAPSO).

More preferably the alkali is aqueous NaOH or aqueous KOH.

In one embodiment the concentration of the alkali solution in the acid-base indicator composition is between about 35 to about 200 mM. Preferably the concentration of the alkaline solution is between about 40 to about 175 mM, more preferably between about 45 to about 140 mM.

The concentration of the alkali in the acid-base indicator composition will mainly determine the pH of the composition before exposure to CO₂. The alkali concentration should be sufficiently high so as to provide a pH of about 10 or above, in the presence of the buffer system. Sensor devices of the invention using different acid-base indicators and different buffer systems will need different amounts of alkali to be present.

In one embodiment the pH of the acid-base indicator composition is greater than about 10.5. Preferably, the pH of the acid-base indicator composition is greater than about 11, more preferably, greater than about 11.5 and most preferably, greater than about 12.

In one embodiment the pH of the acid-base indicator composition is between about 10.5 and about 13.5. Preferably, the pH of the acid-base indicator composition is between about 11 and about 13.5, more preferably, between about 11.5 and about 13.5 and most preferably, between about 12 and about 13.5.

The sensitivity of the sensor device is strongly correlated to the concentration of alkali in the acid-base indicator composition.

Different concentrations of alkali solution cause the devices of the invention to change colour at different CO₂ concentrations, even when the same concentration of the same acid-base indicator is present. Therefore, the concentration of the alkali solution can be used to tune the sensor so that it changes colour at the appropriate CO₂ concentration.

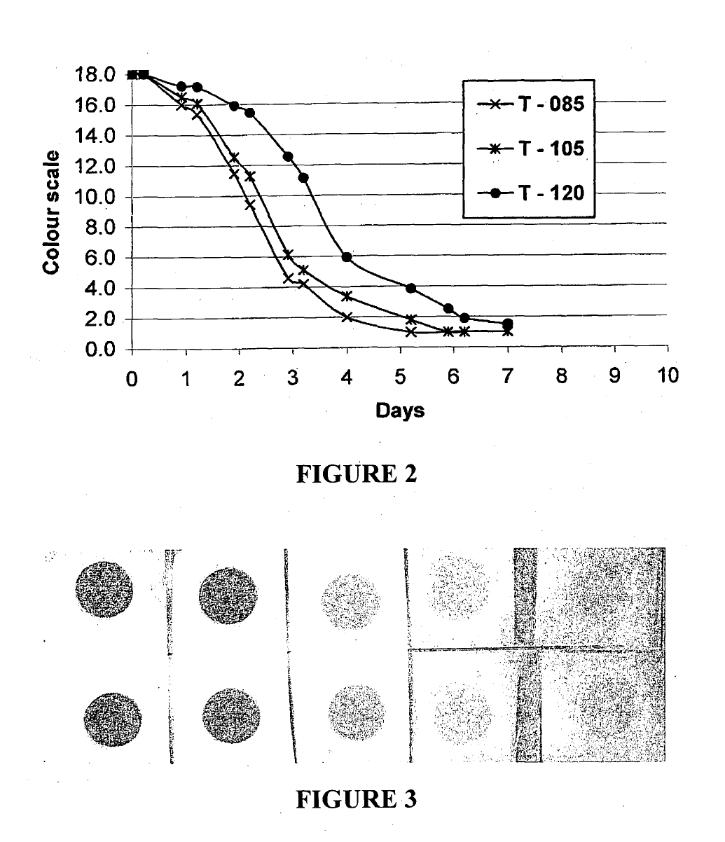

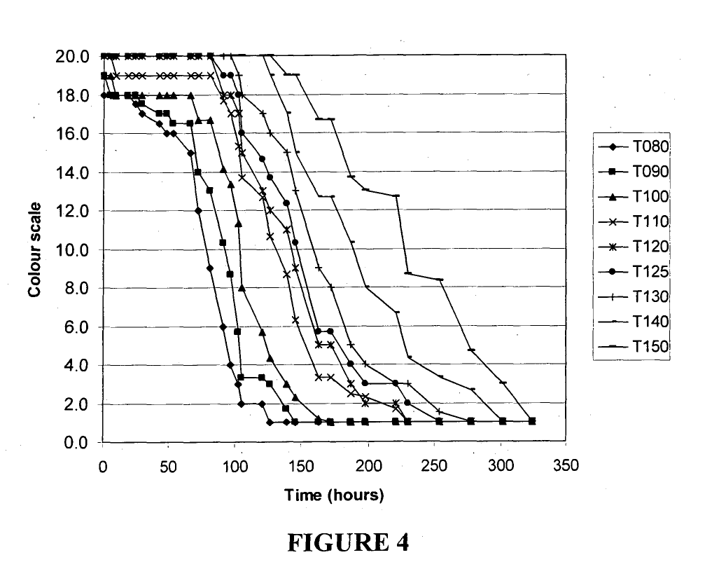

In the Examples provided, sensors are designated T-x where x is the concentration of NaOH in the acid-base indicator composition. Example 1 describes the manufacture of various sensor devices of the invention made using acid-base indicator compositions comprising different concentrations of alkali. As can be seen in FIGS. 2 and 4, sensors comprising acid-base indicator compositions of lower NaOH concentration change colour more quickly in response to the same CO₂ concentration than do sensors comprising acid-base indicator compositions of higher NaOH concentration.

This correlation applies irrespective of the acid-base indicator used, as can be seen in Example 8, as demonstrated in FIG. 11.

In one embodiment the acid-base indicator composition comprises a buffer system.

Preferably, the buffer system comprises at least one buffer selected from the group comprising phosphate buffers, CAPS, tricine, glycine, CAPSO, taurine and 2-(N-cyclohexylamino)-ethanesulfonic acid (CHES).

Preferably the phosphate buffer is disodium hydrogen phosphate dihydrate (Na₂HPO₄.2H₂O) or a salt, such as a potassium salts of phosphoric acid.

In one embodiment the buffer system includes one or more buffers that in combination have at least two pKₐs that between them span the pH range of the acid-base indicator colour change. More commonly the buffer system comprises disodium hydrogen phosphate dihydrate and CAPS.

In one embodiment the concentration of the at least one or more buffers in the acid-base indicator composition is between about 5 mM to about 40 mM, preferably between about 10 mM to about 30 mM, and more preferably between about 10 to about 20 mM.

The buffer system acts with the alkali to keep the pH of the acid-base indicator composition in the appropriate range to ensure a stable colour change in response to exposure to CO₂. In one embodiment the pH of the acid-base indicator composition is greater than about 10. Preferably, the pH of the acid-base indicator composition is greater than about 10.5, more preferably greater than about 11 and most preferably greater than about 12.

In one embodiment the pH of the acid-base indicator composition is between about 10.5 and about 13. Preferably, the pH of the acid-base indicator composition is between about 11 and about 13, more preferably, between about 11.5 and about 13 and most preferably, between about 12 and about 13.

In one embodiment the pH of the acid-base indicator composition is greater than the pKᵢₙ of the acid-base indicator.

In one embodiment the acid-base indicator composition further comprises a salt such as

NaCl or KCl. The salt acts as a preservative. Preferably, the acid-base indicator composition comprises NaCl.

In one embodiment the concentration of the salt in the acid-base indicator composition is between about 20 to about 300 mM. Preferably, the concentration of the salt is between about 50 to about 200 mM, more preferably, between about 75 and 150 mM.

In one embodiment the acid-base indicator composition further comprises a chelating agent. The chelating agent ensures that any heavy metal contamination does not affect the working of the device.

Preferably the chelating agent is ethylenediaminetetraacetic acid (EDTA) or ethylene glycol-bis(2-aminoethylether)-N,N,N¹,N¹-tetraacetic acid (EGTA). Preferably the chelating agent is EDTA.

In one embodiment the concentration of chelating agent in the acid-base indicator composition is between about 1 to about 50 mM. Preferably, the concentration of chelating agent is between about 2 to about 20 mM, more preferably 5 mM.

The acid-base indicator composition may also contain other reagents. For example, the presence of one or more organic solvents may be required to dissolve the at least one acid-base indicator. The organic solvent may also act as a preservative. Organic solvents suitable for use in the invention are miscible with water and include ethanol, methanol, acetone, isopropyl alcohol, ethylene glycol, propylene glycol, dimethylformamide, dimethylsulfoxide, acetonitrile, and the like.

In one embodiment the concentration of organic solvent in the acid-base indicator composition is between about 0 to 20% v/v, preferably between about 5 to 25%, more preferably 10%.

In one embodiment the acid-base indicator composition contains ethanol. Preferably, the concentration of ethanol is between about 0 to 20% v/v, more preferably, about 10% v/v.

The acid-base indicator composition may optionally also contain humectants to prevent it from drying out completely. While some of the solvent may evaporate out of the acid-base indicator composition, some water must remain so that the acid-base indicator can change colour. Suitable humectants include glycerol, sorbitol, maltitol, polydextrose, triacetylglycerol, propylene glycol, sodium lactate, potassium lactate, isomalt, xylitol and the like.

In one embodiment the acid-base indicator composition comprises glycerol.

The acid-base indicator composition may also comprise antifreeze agents such as ethylene glycol to prevent the solution freezing at low temperatures.

In embodiments of the invention where the device is placed in direct contact with the food, the reagents present in the device must be non-toxic.

The reagents present in the acid-base indicator composition and their relative concentrations are selected to change colour at the desired CO₂ concentration. Accordingly, the devices of the invention can be ‘tuned’ to have different sensitivities depending on the application for which they are intended.

For example, different fruits will emit different amounts of CO₂ in the course of achieving ripeness. When assessing the ripeness of fruit that emit a large amount of CO₂ early in the ripening process, the sensor devices of the invention can be tuned to indicate ripeness only once the CO₂ concentration becomes relatively high.

In contrast, microbial food spoilage may occur much more quickly. Therefore, sensor devices of the invention intended for monitoring CO₂ from food dangerous spoilage micro-organisms may need to be tuned to change colour in the course of a few hours when only a small amount of CO₂ has been emitted.

The acid-base indicator compositions for use in the sensor devices of the invention change colour stably. This stable colour change ensures that the quantities of CO₂ emitted, and therefore the ripeness of the fruit, degree of microbial activity and the like can be accurately determined.

If the colour change is not stable, the colour of the sensor device will indicate only the concentration of the CO₂ at the time of reading. However, for a sensor device to be useful it needs to provide a measure of the cumulative CO₂ that has been emitted over a given time period.

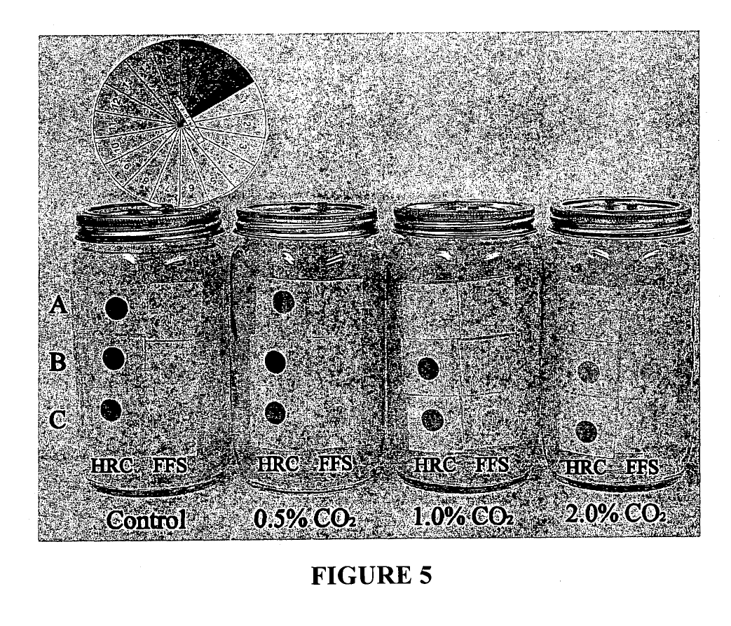

The irreversible properties of the sensor devices of the invention are investigated in Example 5. Sensor devices of three sensitivities, T-045, T-087.5 and T-140 were prepared each using one of three laminate films, A1, A2 and A3, as described in Example 4. The performance of these sensor devices (the HRC sensors) was compared to sensor devices made in accordance with PCT application WO 2006/062870 (the FFS sensors). All of the sensors were exposed to different CO₂ concentrations for 44 hours at room temperature. As can be seen in FIG. 5 all of the sensor devices changed colour—the HRC sensors from red to yellow, and the FFS sensors from pale yellow to green.

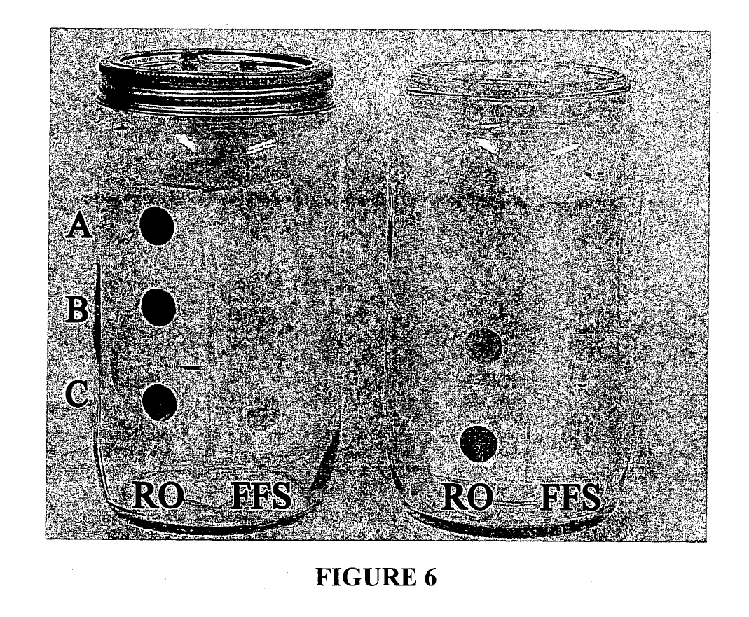

When the CO₂ concentration was decreased by exposing the sensors to the open air, the HRC sensors remained the same colour whereas the FFS sensors reverted to their original colours (see FIG. 6).

These results demonstrate that the sensor devices of the invention stably change colour.

A reversible sensor device such as the FFS sensor will be unreliable. Decreases in CO₂ concentration of the environment of the sensor, such as may occur when packaging is opened, will cause the sensor device to revert to its original colour. Therefore, the sensor will not provide any useful information, for example regarding the ripeness of the fruit or the degree of microbial activity.

The irreversibility of the sensor devices of the invention was also demonstrated in Example 8 where sensor devices using different acid-base indicators were used. All of the devices provided a stable colour change.

### 4. The Support Base and Viewing Cover

The components of the sensor devices of the invention comprise
(a) a support base;
(b) an acid-base indicator composition; and
(c) a viewing cover.

The acid-base indicator composition is disposed between the support base and the viewing cover. In one embodiment the acid-base indicator composition is disposed within a carrier matrix.

The carrier matrix can be any known art substance capable of incorporating the acid-base indicator composition for use in the invention provided that the carrier matrix is also substantially inert with respect to the reagent. The carrier matrix is porous or absorbent relative to the reagent.

In one embodiment the carrier matrix is selected from the group comprising filter paper, chromatographic paper, porous membranes, sponge material, paper, cellulose, wood, woven or non-woven fabric, polymeric film, glass fibre, silica gel, alumina, and the like. Preferably, the carrier matrix is filter paper such as Phase Separation (PS) paper (Smith Scientific, Kent, England)

The support base and the viewing cover may be made of the same material or different materials, provided that at least one of the support base or the viewing cover is CO₂ permeable. The viewing cover must contain a transparent or partially transparent portion such that the colour change of the acid-base indicator composition can be observed. In one embodiment the whole viewing cover is transparent.

The support base may be transparent but can also be opaque. The support base and the carrier matrix may be separate or integrally formed.

The sensor device of the invention can be made by enclosing the acid-base indicator composition between a support base and the viewing cover.

In one embodiment the support base is CO₂ permeable and the viewing cover is CO₂ impermeable.

In another embodiment the support base is CO₂ impermeable and the viewing cover is CO₂ permeable.

In another embodiment the both the support base and the viewing cover are CO₂ permeable.

In one embodiment the CO, permeable layer allows transmission of at least 4 cm³/m²/hr CO₂ at 23° C.

When in use, CO₂ emitted from a CO₂ emitting source near to the sensor device passes through the CO₂ permeable layer formed by the support base, the viewing cover or both, to the acid-base indicator composition disposed between them. Reaction of the CO₂ with the components of the acid-base indicator composition causes the acid-base indicator composition to change colour, by decreasing the pH of the composition. When the sensor device is removed from the source of CO₂, CO₂ may diffuse out of the acid-base indicator composition through the CO₂ permeable support base, viewing cover, or both. However, the acid-base indicator composition does not change colour in response to the decrease in CO₂ concentration, as discussed above.

As the reader will appreciate, the support base and the viewing cover can be made from a wide range of suitable materials known in the art.

In the sensor device of the invention at least one of the support base or the viewing cover is CO₂ permeable. CO₂ permeable laminate films are known in the art and are described in JP-A-5-222215, JP-A-9-316208, JP-A-11-538 and U.S. Pat. No. 6,316,067 incorporated herein by reference.

In one embodiment the support base and/or the viewing cover are made of laminate film. Preferably, the laminate film is selected from the group comprising standard thermal laminating film, low temperature thermal laminating film, heatset (or heat assisted) laminate films, pressure sensitive film, liquid laminates and mixtures thereof.

Types of laminate films suitable for use in the sensor devices of the invention include polypropylene (PP) laminates including orientated PP laminates, biaxially orientated PP laminates, white matt PP laminates, transparent matt PP laminates, clear glossy PP laminates; polyvinyl chloride (PVC) laminates; polyolefin laminates; polyethylene (PE) laminates; including low density, medium density, high density, linear low density and polyethylene vinyl acetate (EVA) laminates, white matt PE laminates, and the like.

In one embodiment the viewing cover comprises a laminate film as described above.

In one embodiment the support base comprises material selected from paper, cardboard, fabric, metal such as aluminium foil and mixtures thereof. In another embodiment the support base comprises laminate film. In a further embodiment the support base comprises laminate film, and a backing material selected from paper, cardboard, fabric, metal such as aluminium foil and mixtures thereof.

The laminate film may comprise one or more layers. The devices of the invention may be prepared by any suitable methods of lamination known in the art.

The nature of the laminate film may also influence the sensitivity of the sensor devices of the invention. Example 4 describes the manufacture and testing of sensor devices of the invention made using different laminate films.

The laminate films used were polypropylene and polyethylene laminate films. The colour change response of the sensors varied with the type of laminate used, as can be seen in Table 2.

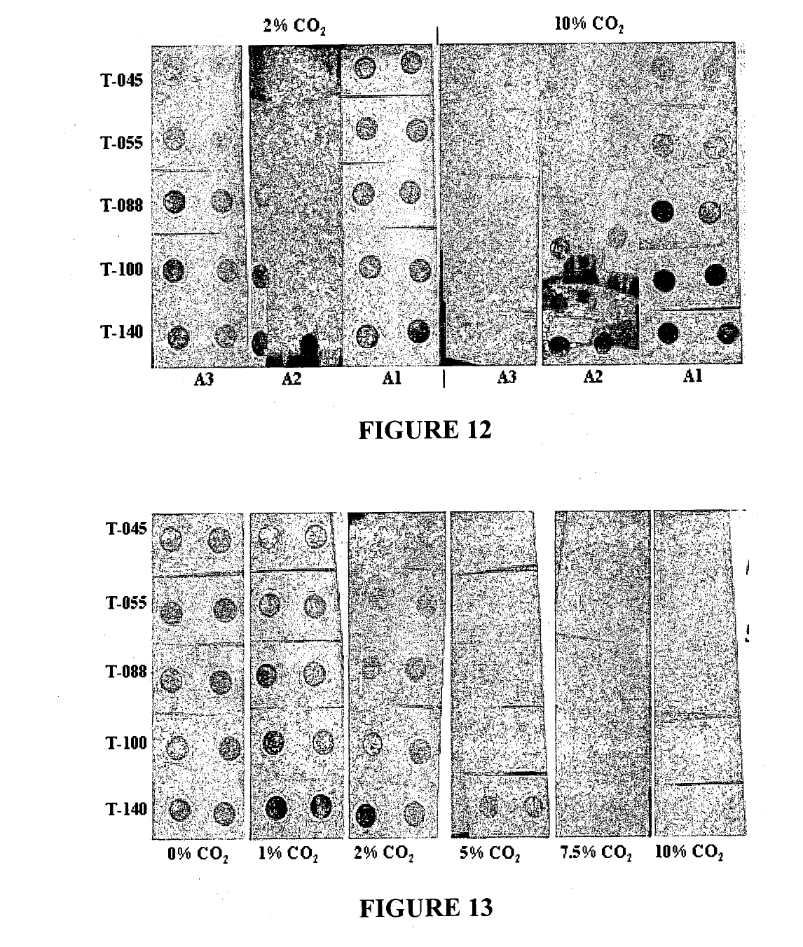

The types of laminates for use in the invention were also investigated in Example 9. Sensor devices of five different sensitivities were made, each using one of three different laminate films. The results, shown in Table 6 and FIGS. 12 and 13, indicate that the colour change of the sensor devices of the invention is influenced both by the sensitivity of the acid-base indicator composition (T-x) and the type of laminate film used.

Therefore, the type of laminate film selected can, along with the concentration of the alkali, be used to tune the sensor device to a particular application. The person skilled in the art will be able to select an appropriate laminate film so that the sensor device changes colour at the appropriate time for the application.

In one aspect the invention provides a device for detecting CO₂ comprising:
(a) a support base,
(b) an acid-base indicator composition of pH greater than 11, and
(c) a viewing cover
   wherein at least one of (a) and (c) is CO₂ permeable, and wherein (b) is disposed between (a) and (c) such that the colour change of (b) can be observed through (c), and wherein the acid-base indicator composition comprises Alizarin yellow R, NaOH, disodium hydrogen phosphate dehydrate buffer and CAPS.

In one aspect the invention provides a device for detecting CO₂ comprising an acid-base indicator composition that stably changes colour upon exposure to CO₂ wherein the acid-base indicator composition is disposed between two layers of laminate film.

In one embodiment the laminate film is selected from polypropylene and polyethylene film.

In one embodiment the acid-base indicator composition comprises Alizarin yellow R, NaOH, ethanol, glycerol, NaCl, disodium hydrogen phosphate dehydrate buffer, CAPS, and EDTA.

### 5. Methods of Using the Sensor Devices of the Invention

The sensor devices of the invention can be used for detecting CO₂ from many CO₂ emitting sources. The emission of CO₂ can be correlated to many processes to provide useful information.

In one aspect the invention provides a method for indicating fruit ripeness using a sensor device of the invention. In another aspect the invention provides a method for indicating food spoilage using a sensor device of the invention. In another aspect the invention provides a method for indicating food fermentation using a sensor device of the invention. In another aspect the invention provides a method for indicating CO, emissions from soil using a sensor device of the invention.

In use, the sensor device of the invention is placed near the CO₂ emitting source, for example fruit, food or soil. As used herein the terms “near the fruit” or “near the food” mean that the device is placed at a distance that allows accurate and reliable detection of CO₂ being emitted from the fruit or food. Preferably, the device is placed on or up to about 10 cm from the fruit, food or soil.

The device of the invention can be applied to a fruit or food item by any suitable means. For example, the device can be in the form of a patch or label which can be adhered directly to the fruit or food item. A particularly convenient apparatus which can be used is a labelling machine of the kind described in European Patent EP 0113256.

If placed directly on the fruit or food, the support base may be partially covered with adhesive, to allow the device to remain in contact with the fruit or food. In this embodiment the support base may be CO₂ permeable to allow direct diffusion of CO, into the sensor device.

The device of the invention may also be incorporated into the packaging surrounding the fruit or food. The packaging may include an individual item of fruit or food, or several items.

The sensor device of the invention and the CO₂ emitting source need not be placed in a closed system so that no CO₂ can escape and no other gases can enter. However, the transfer of gases from the area in which the sensor device and CO₂ emitting source are placed should be relatively low.

The sensor devices of the invention can be tuned to change colour at particular CO₂ concentrations. A highly sensitive device would be more suitable for use in low CO₂ environments, such as where gas exchange in the area of the device and CO₂ emitting source is high. For example, where the sensor device of the invention is packaged with fruit in a substantially open container.

CO₂ is produced by fruit at all stages of ripening but fruit that demonstrate a noticeable burst of CO₂ production during ripening are referred to as climacteric fruit. Kiwifruit, pears and avocados are examples of climacteric fruit. The production of CO₂ can be correlated to the ripeness of climacteric fruit.

In one embodiment the device of the invention is particularly suited for detecting fruit ripening in climacteric fruit. Preferably the fruit is selected from the group comprising avocados, climacteric stonefruit (including apricot, peach, nectarine, plum and the like), pears, melons, mangos, papaya, bananas, apples, kiwifruit, and paw paw. More preferably, the fruit is kiwifruit or avocado.

The climacteric process can proceed while the fruit is attached to the plant, or after detachment. For example, kiwifruit are normally harvested before they are ripe. The ripening process continues after the fruit has been detached from the plant. Therefore, the device of the invention can be used to indicate fruit ripeness while the fruit is still attached to the plant, or after harvesting.

More commonly, the sensor device of the invention is packaged and sold with the fruit to provide the distributor and consumer with information about the ripeness of the fruit.

The sensor devices of the invention are suitable for use with most types of fruit packaging including trays, bags, boxes, flow wrapping, cartons, punnets and “clamshells”. The packaging may be vented and can be made of any material such as plastic, paper, paper pulp, cardboard or the like, providing that the overall arrangement and permeability of the packaging is such that the CO₂ concentration can build up to an appropriate level—approximately 0.5-4%. In one embodiment the CO₂ concentration can build up to about 0.5 to 2.0%.

The sensor devices of the invention can be secured to the packaging by any means known in the art. In one embodiment the sensor devices are glued or taped to the underside of the transparent packaging, so that the viewing cover can be seen through the packaging without opening the packaging. In another embodiment the sensor devices are integrated into the packaging itself. In another embodiment the sensor devices are placed with the fruit in the packaging unsecured.

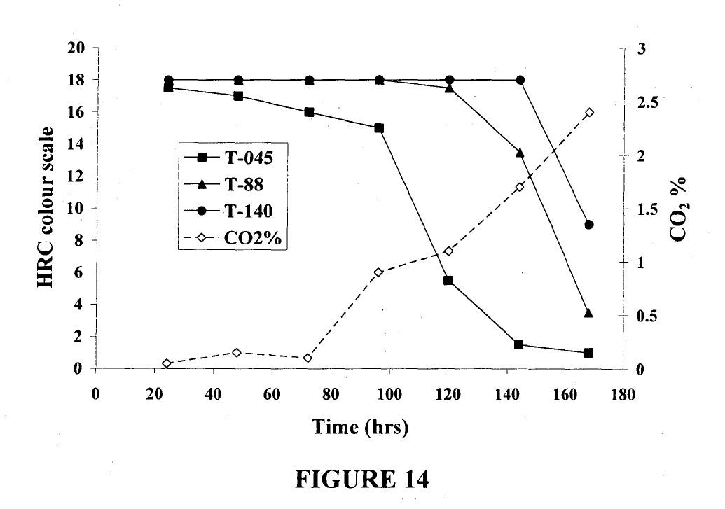

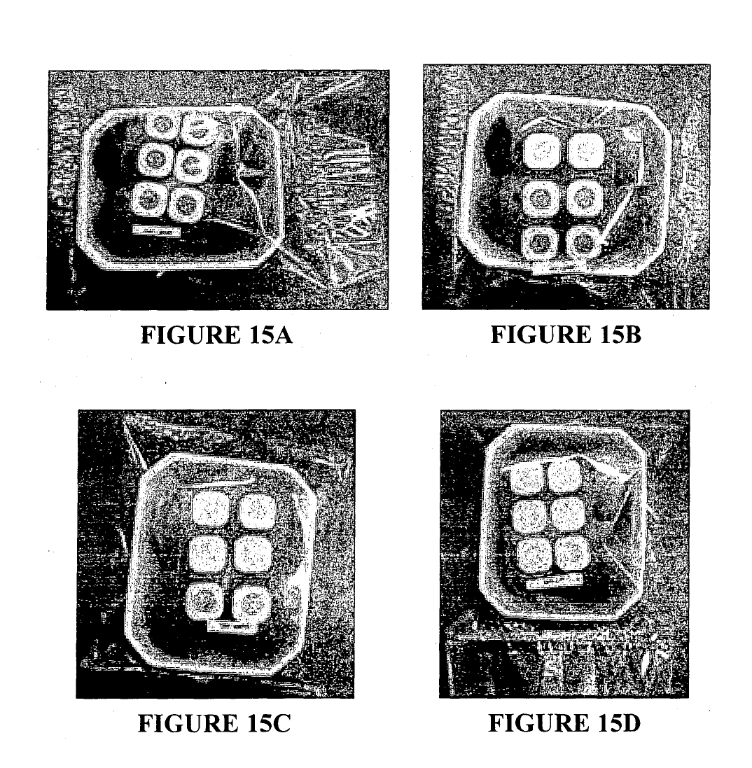

The use of the sensor devices of the invention with flow wrapping packaging is demonstrated in Example 10. Packaged kiwifruit were left at room temperature for 168 hours. The results are shown in FIGS. 14 and 15. The sensor devices of the invention were able to correlate ripeness of the kiwifruit with the colour change.

Micro-organisms present in food also produce CO₂ during food spoilage and fermentation. Therefore, the sensor device of the invention can also be used to detect CO₂ emitted by food micro-organisms.

In one embodiment the sensor device of the invention is particularly suited for indicating food spoilage caused by one or more species of micro-organisms including comprising Salmonella enteriitidis, Campylobacter jejuni, Escherichia coli including 0157:H7, Staphylococcus aureus, Shigella dysenteriae, Listeria monocytogenes, clostridium peringen, Bacillus cereus, Vibrio parahaemolyticus, Vibrio cholerae, Yersinia enterocolitica, Brochrothrix thermosphacta, Pseudomonus sp., Acromonas sp., Lactobacillus plantarum, Lactobacillus fructivorans, Serratia liquefaciens, and Zygosaccharomyces bacilli.

The device of the invention can be used to indicate food spoilage in foods selected but not limited to the group comprising meat, fish, seafood, eggs, poultry, grains, flour and cereal products, legumes, rice, dairy products such as milk, cheese, cottage cheese, yoghurt, cream and fruit juices.

As in the methods of detecting fruit ripening, to detect food spoilage the sensor device of the invention should be placed near to the food, preferably in an environment that allows the build up of sufficient CO₂ to achieve a colour change at the appropriate time.

If present in the packaging, the device can be incorporated into a milk bottle or carton, a bottle cap, a wine stopper, plastic wrap, plastic box, styrofoam, plastic bag, cardboard, paper, metal foil and the like.

To detect spoilage of milk, the sensor devices of the invention can be incorporated into the milk packaging, preferable in the lid of the package. Even when packaging is opened repeatedly, the CO₂ concentration in the package increases sufficiently quickly so that the sensor devices change colour when the milk micro-organisms are at an unsafe level.

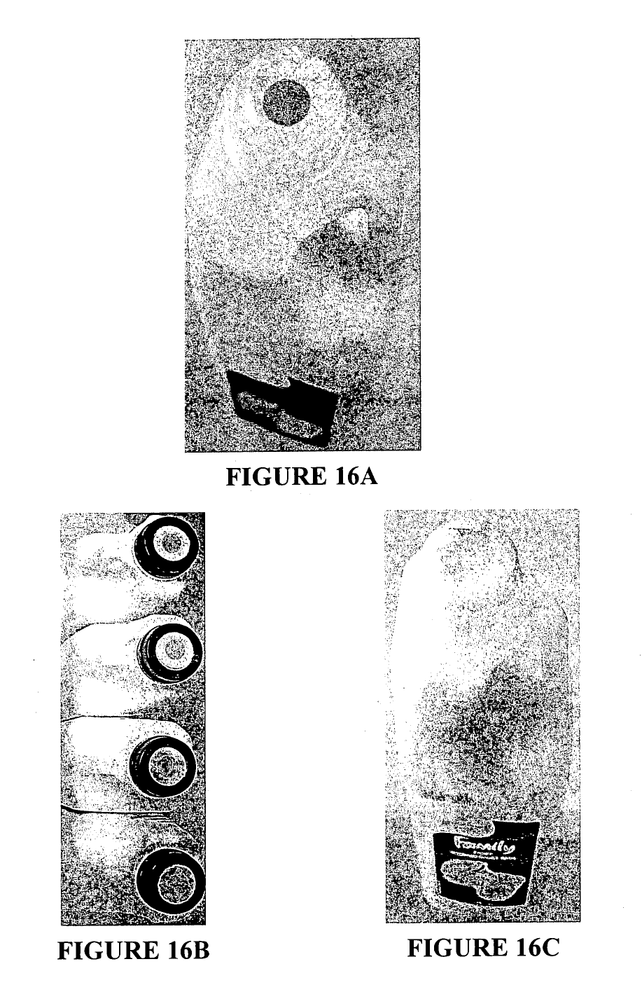

FIG. 16 shows a sensor device incorporated into a plastic milk bottle top, and the colour change that results as the milk spoils.

The sensitivity of the sensor device may be altered to accommodate the type of food product. For example, full cream requires a sensor device of a different sensitivity to low fat milk.

In another embodiment the device of the invention is particularly suited for indicating food fermentation caused by one or more species of micro-organism including but not limited to the group comprising Lactocooccus lactis, Steptococcus thermophilus, Leuconostoc sp., Pediococcus sp., Lactobacillus sp., Bifidobacterium sp., Propiopribacterium sp., Lactobacillus delbruekii, Streptococcus thermophilus, Saccharomyces cerevisiae, Candida rugosa, Kluyveromyces marxianus, Aspergillus oryzae, and the like.

The device of the invention can be used to indicate fermentation of foods selected but not limited to the group comprising acidophilus milk, amasake, beer, bleu cheese, bread, cheese, chocolate, cider, coffee, cultured vegetable, kefir, kimchi, kombucha, kumiss, marinated vegetables such as artichokes and mushrooms, mahewu, miso, nuoc mam, olives, pickles, quorn, sauerkraut, sourdough bread, soy sauce, tea, tempeh, umeboshi plums, vinegar, wine, yoghurt, cottage cheese, bologna, salami, tofu, and sour cream.

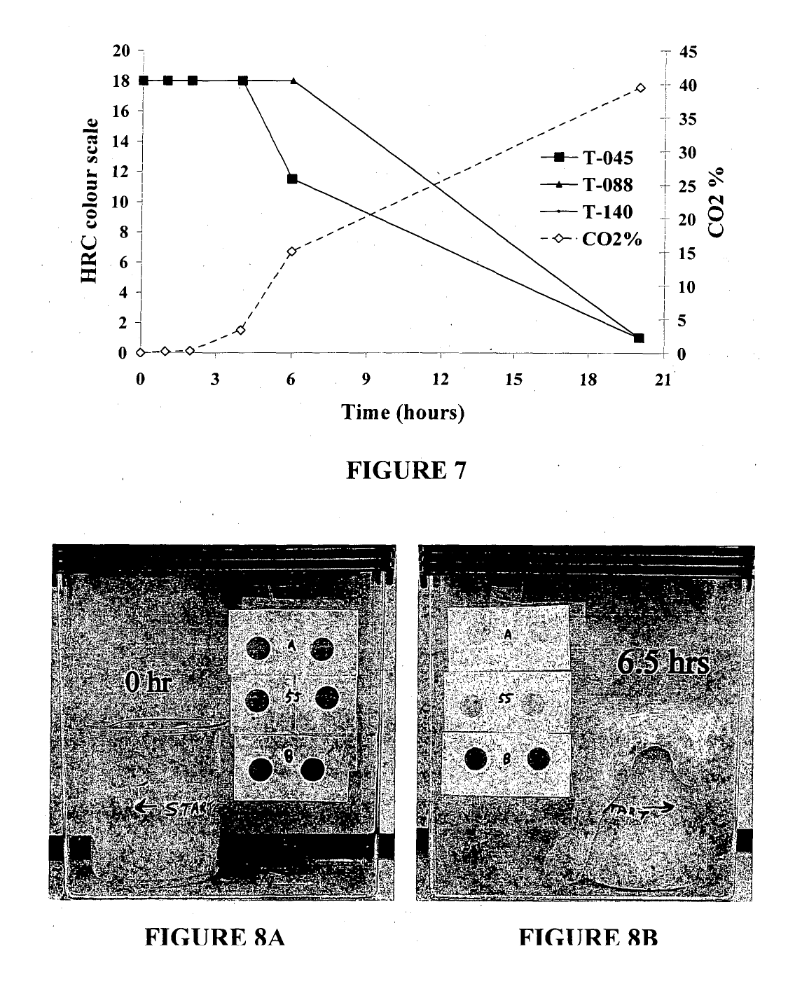

Example 6 demonstrates how the sensor devices of the invention can be used for monitoring food fermentation. In Example 6 the sensor devices indicated the fermentation of flour dough by yeast. The results are shown in FIGS. 7 and 8.

Use of a sensor device of the invention could provide quality assurance of the finished products. For example, in the case of flour dough, the quality of the final product could vary with the yeast, the source of flour and variations in the fermentation conditions. The sensor devices could be used in the pizza and bread industries to monitor fermentation until a pre-determined point as indicated by the sensor colour. They may also be used to monitor fermentation in wine and beer making.

In use, the sensor devices of the invention could be fixed to the inside of a transparent fermentation vessel. Alternatively, the sensor device could be attached to the end of a probe, where the probe is designed to be inserted into the dough or other fermenting food, the CO₂ travels through the probe to the sensor device at the other end.

The sensor devices of the invention can also be used to measure CO₂ loss from the soil due to soil micro-organisms. Soil micro-organisms responsible for CO₂ emissions include free living bacteria and other microbes and nitrogen-fixing bacteria present in root nodules of clover and other legumes.

CO₂ emissions from soil can be measured using electronic instruments (for example, “Automatic soil CO₂ exchange system” ADC BioScientific, Herts, England) but these are very expensive. Measurements of CO₂ emissions are also indicative of soil microbial activity. In pastures containing nitrogen-fixing plants such as clover in a rye-glass clover pasture and the like, the activity of the nitrogen-fixing bacteria is linked to the health of the clover plant, and will dictate the amount of nitrogen fixed by the plant.

Therefore, the sensor devices of the invention may provide information about how much nitrogen fertiliser is required for a particular area. In use, the sensor device of the invention could be placed on one end of a probe and the other end of the probe inserted into the soil near the rhizosphere of the nitrogen-fixing plants.

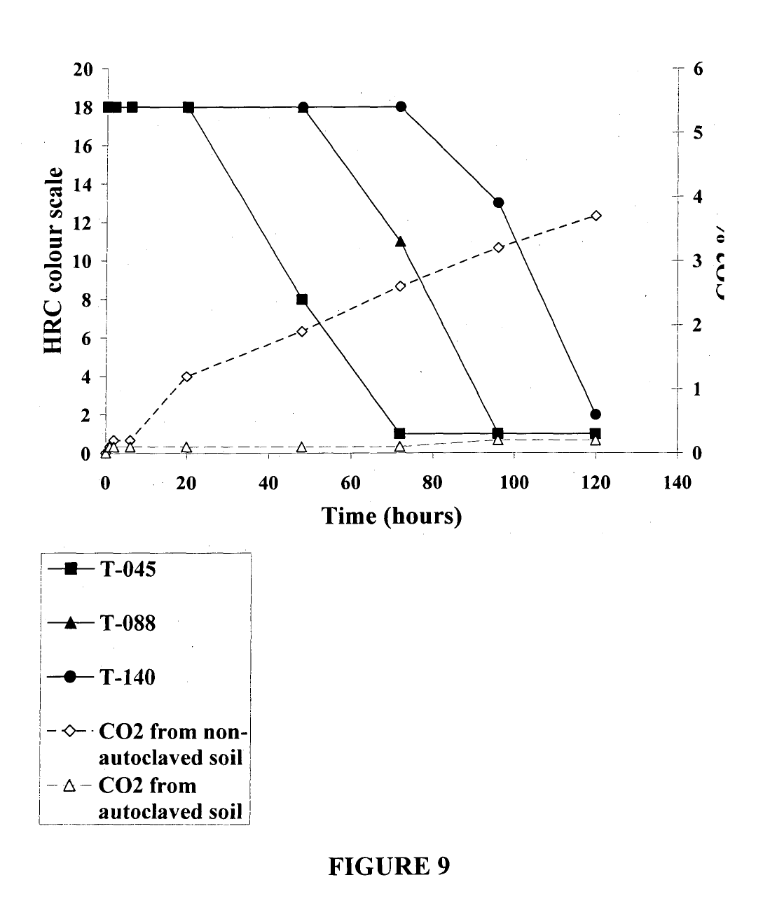

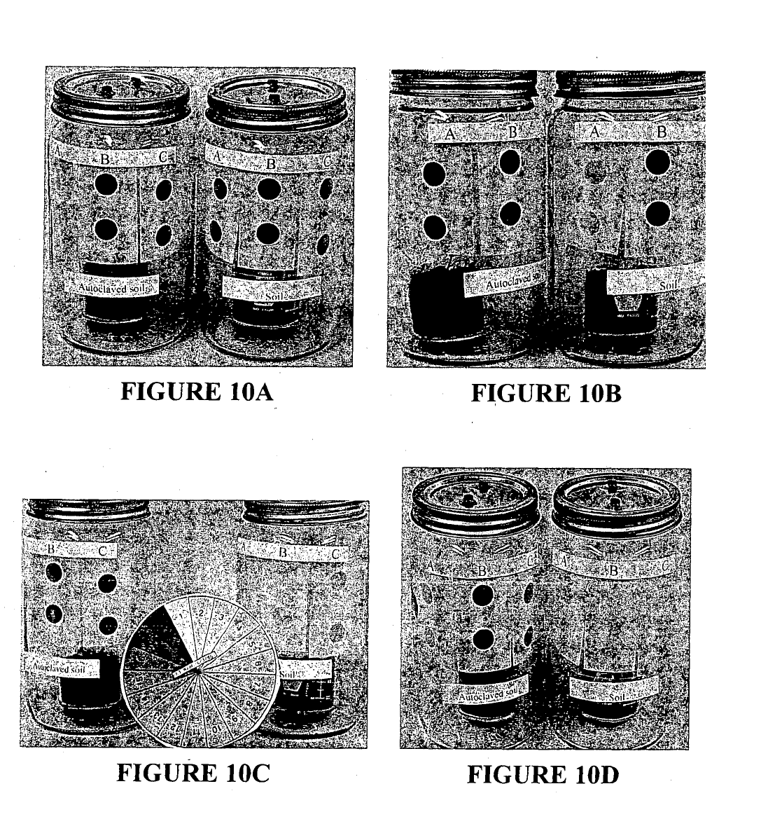

Example 7 investigates the measurement of CO₂ from the soil. The results are shown in FIGS. 9 and 10.

In the methods of the invention, the colour changes are quantified by comparing the sensor colour against a set of standard colours between red (denoted by number 18) and bright yellow (denoted by 1). In between colours representing various shades of red, orange and yellow were denoted by numbers 17 to 2. The colour wheel as shown in FIGS. 5 and 10 was developed by Dr. Bob Jordan at HortResearch in March, 2006.

The colours in the wheel have also been calibrated using Spectrometer (StellarNet Inc; Model Number-EPP2000-NIR-200). The “L”, “a” and “b” values for colour numbers 18, 12, 5 and 1 are shown in the table below.

TABLE 1
“L-a-b” values for colour wheel as shown in FIG. 1.

| Colour Scale from | Mean ± Standard Deviation | | |
|---|---|---|---|
| wheel | L* | a* | b* |
| 18 | 15.4 ± 0.04 | 35.8 ± 0.17 | 7.8 ± 0.59 |
| 12 | 23.2 ± 0.01 | 26.9 ± 0.14 | 14.6 ± 0.48 |
| 5 | 38.8 ± 0.01 | 14.2 ± 0.07 | 27.2 ± 0.26 |
| 1 | 43.9 ± 0.02 | 1.6 ± 0.08 | 25.5 ± 0.22 |

In future, the 18 points wheel may be condensed to 8 points covering only the major shades of red, orange and yellow.

For the device of the invention to provide an accurate indication of fruit ripeness based on cumulative CO₂ release, it is necessary to first know the state of ripeness of the fruit at the time the device is first introduced into the vicinity of the fruit. In addition, the quantity of CO₂ typically released as the fruit progresses from this point to full eating ripeness must also be known. These values can be determined using standard methods in the art.

Time spent in storage, storage conditions such as temperature, humidity etc and postharvest treatments (eg ethylene conditioning) are all known to influence the rate of ripening. Therefore, these factors should be considered when tuning a device of the invention for a particular application.

Analogous factors apply to the detection of CO₂ emitted by food or other types of micro-organisms.

The combination and concentrations of the reagents in the acid-base indicator composition are tuned to ensure that the colour change in response to CO₂ is stable. Accordingly, once the sensor device indicates that a certain amount of CO₂ has been emitted from the fruit or food, later diffusion of CO₂ out of the sensor device will not cause the colour of the acid-base indicator composition to change.

### 6. Kits Comprising the Sensor Device of the Invention

The invention also provides kits comprising a CO₂ detecting sensor device of the invention and a colour chart correlating the possible colours of the acid-base indicator composition with the parameter to be measured. For example, in kits for indicating fruit ripeness where the colour changes from red to yellow, the colour chart may show a range of colours from red, through orange, to yellow. The colour chart may also include wording that correlates the colour to the degree of ripeness, for example yellow meaning ripe, orange meaning partially ripe and red meaning unripe.

Similarly, kits for indicating food spoilage or food fermentation may include colour charts linking the colour of the sensor device with the degree of food spoilage or fermentation.

Various aspects of the invention will now be illustrated in non-limiting ways by reference to the following examples.

### Example 1

CO₂ sensor devices of various sensitivities were prepared by following a four step protocol as outlined below. The number after “T” in the sensor type designation refers to the concentration (in mM) of NaOH in the acid-base indicator composition, i.e., the concentration of NaOH in the reagent solution after dilution with dye solution (see Step 3).

### Step 1-Preparation of Reagent Solution

| | Volume (ml) of stock solutions of various components | | | | | | |
|---|---|---|---|---|---|---|---|
| | | 0.25 M | | | | Steri- | |
| | | Disodium | | | | lized | Total |
| Sensor | 0.5 M | hydrogen | 0.5 M | 2 M | 20 mM | MilliQ | Vol- |
| types | NaOH | phosphate | CAPS | NaCl | EDTA | water | ume |
| T-085 | 3.40 | 0.8 | 0.2 | 1.0 | 1.0 | 3.60 | 10 |
| T-105 | 4.20 | 0.8 | 0.2 | 1.0 | 1.0 | 2.80 | 10 |
| T-120 | 4.80 | 0.8 | 0.2 | 1.0 | 1.0 | 2.20 | 10 |

For long term storage the reagent solutions should be stored in airtight plastic tubes in the dark.

### Step 2-Preparation of Dye Solution

Dye solution was prepared by dissolving 16 mg of Alizarin yellow R (CAS No 1718-43-9, Acrus Organics, New Jersey, USA) in 10 ml of 20% ethanol (v/v)+20% (−2.5 g) glycerol (v/v) in sterile MilliQ water. Most of the dye is solubilised immediately. Traces of insoluble dye particles are solubilised during overnight incubation. The dye solution is stored in the dark. The concentration of Alizarin yellow R in this solution is 5.17 mM.

### Step 3-Preparation of Acid-Base Indicator Composition

The reagent solution and the dye solution prepared in steps 1 and 2 respectively, were mixed in equal volumes in a plastic container. The tube was tightly capped, covered by aluminum foil and stored in dark.

### Step 4-Preparation of Device for Detecting CO₂

The acid-base indicator composition prepared in step 3 was loaded onto filter paper. The filter paper was then laminated between a CO₂ permeable support base and a CO₂ permeable viewing cover.

### Example 2

### Typical Response of Sensor Devices to CO₂

The transparent side of the laminated sensor devices made in Example 1 (types T-085, T-105 and T-120) was stuck to CO₂ impermeable clear plastic sheet and then placed in a glass jar containing 1% CO₂. The jars were incubated at ˜20° C.

The CO₂ gas permeated through the CO₂ permeable support base. The sensor colour changed from red to yellow via orange. The colour change was monitored using an arbitrary scale of 1 (bright yellow) to 18 (deep red). The timing of the onset of colour change and the slope of the colour change depended on the composition of the sensor solution.

FIG. 2 shows the colour response of three sensor devices of different sensitivities when incubated at room temperature in the presence of 1% CO₂. The data are the average of 12 measurements (4 sensor devices in 3 jars).

FIG. 3 shows the actual colour change of three sensor devices of the invention after exposure to 1% CO₂. The starting colour is on the left. The sensor devices change from red to yellow from left to right.

### Example 3

The colour change in response to CO₂ was measured for nine sensor devices of the invention T-085-T-150 under the conditions outlined in Example 2. The value of “T” refers to the concentration of NaOH in mM.

The results are shown in FIG. 4. The sensor devices are tuned to have different sensitivities to CO₂. The data points are the means of three values. The colour change was measured using an arbitrary scale of 1 (bright yellow) to 20 (deep red).

This experiment demonstrates that the sensor devices of the invention can be tuned to change colour in response to CO₂ slowly or quickly, be changing the concentration of reagents in the acid-base indicator composition.

### Example 4

The effect of different types of laminates was investigated by preparing nine sensor devices of the invention, using three different laminates.

The sensor devices of the invention were made using the protocol outlined in Example 1 except that the sensors differed in their NaOH concentration. T-045, T-088 and T-140 sensors were prepared using different types of laminate film for the support base and viewing cover. A1 is a polypropylene laminate film. A2 is an orientated polypropylene laminate film and A3 is a medium density polyethylene laminate film.

All of the sensors were exposed to air containing various percentages of CO₂-0% (air), 0.5%, 1.0% and 2.0% for 44 hours at room temperature. The CO₂ was added to a sealed jar and its concentration determined using a CheckPoint O₂/CO₂ meter (PBI Dansensor, Ringstead, Denmark).

The colour change in the sensors of the invention after 20 hrs in 2.0% CO₂ is shown in Table 2 below.

TABLE 2

| | Colour | | |
|---|---|---|---|
| Laminate | T-045 | T-088 | T-140 |
| A1 | 12 | 18 | 18 |
| A2 | 5 | 15.5 | 18 |
| A3 | 1 | 2 | 6 |

The numbers represent the colour in the colour wheel as shown in FIG. 5. The colour wheel provides a set of standard colours between red (denoted by number 18) and bright yellow (denoted by 1). In between colours representing various shades of red, orange and yellow are denoted by numbers 17 to 2. The colour wheel was developed by Dr Bob Jordan at HortResearch in March 2006.

The colours in the wheel can be calibrated using Spectrometer (StellarNet Inc; Model Number-EPP2000-NIR-200) The “L”, “a” and “b” values for colour numbers 18, 12, 5 and 1 are shown in Table 3 below.

TABLE 3

| | |
|---|---|
| Colour scale from | Mean ± Standard Deviation |

| wheel | L* | a* | b* |
|---|---|---|---|
| 18 | 15.4 ± 0.04 | 35.8 ± 0.17 | 7.8 ± 0.59 |
| 12 | 23.2 ± 0.01 | 26.9 ± 0.14 | 14.6 ± 0.48 |
| 5 | 38.8 ± 0.01 | 14.2 ± 0.07 | 27.2 ± 0.26 |
| 1 | 43.9 ± 0.02 | 1.6 ± 0.08 | 25.5 ± 0.22 |

It is clear from the above data that the response to CO₂ exposure varied with laminate (sensitivity decreasing order A1>A2>A3). Sensitivity to CO₂ exposure also depended on the T value which reflects the concentration of alkali in the acid-base composition. T-45 was more sensitive, followed by T-088, irrespective of the laminate film used. Similar results were obtained for the sensor devices of the invention exposed to 0.5% and 1.0% CO₂. The sensors exposed only to air (0% CO₂) did not change colour.

### Example 5

To demonstrate the irreversibility of sensor devices of the invention the colour response of the nine sensors described in Example 4 were tested against sensor devices as described in PCT publication WO2006/062870 (FFS sensors).

The FFS sensors were prepared in accordance with the detailed description of WO2006/062870.

Three sets of FFS sensors were prepared each using different laminate films of types A 1, A2 and A3. The composition of the liquid placed between the laminate film is provided in Table 4 below.

TABLE 4
Final concentrations

| | Bromothymol Blue, | Methyl Red, | Ethanol, | NaOH |
|---|---|---|---|---|
| Name | % (w/v) | % (w/v) | % (w/v) | (mM) |
| FFS-A | 0.02 | 0.001 | 10 | 0.5 |
| FFS-B | 0.05 | 0.0035 | 10 | 1 |
| FFS-C | 0.08 | 0.005 | 10 | 1.5 |

The FFS sensors change from a generally green colour to a generally orange colour when exposed to 0.5% CO₂.

All of the sensors were exposed to air containing various percentages of CO₂±0% (air), 0.5%, 1.0% and 2.0% for 44 hours at room temperature as described in Example 4.

The results for the A1 sensors of the invention and the FFS sensors are shown in FIG. 5. Each jar contains sensors of the invention shown as “RO” from top to bottom T-045, T-087.5 and T-140 and FFS sensors, from top to bottom, FFS-A, FFS-B and FFS-C. As can be seen in the control (0% CO₂) only FFS-C was a distinct green colour. The FFS sensors of lower NaOH concentration were either yellow (FFS-A) or very pale green (FFS-B).

The qualitative responses followed a parallel pattern. Upon exposure to CO₂ the FFS sensors changed from a green or yellow/green colour to a yellow or orange/yellow colour. The sensors of the invention changed from bright red to yellow/orange, depending on the sensitivity with T-045 turning bright yellow in 2% CO₂ while T-140 remained red/yellow.

FIG. 5 shows only the changes for A 1 sensor devices, but similar colour changes were observed when A1 laminate film was replaced with the A2 or A3 laminate film.

FIG. 6 shows the colour changes that occurred when sensors exposed to CO₂ were opened to the air, allowing the CO, to dissipate. FIG. 6 shows the “control” and 2% CO₂ jars from FIG. 5, after the lid has been removed from the 2% CO₂ jar for 24 hrs.

While the sensor devices of the invention remain the same colour they were after 44 hrs in 2% CO₂, the FFS sensors have reverted back to the colour they were in the absence of 2% CO₂. Therefore, while the sensors of the invention are irreversible, the FFS sensors are not.

The same results were obtained for the sensor series using the A2 and A3 laminates.

### Example 6

The sensors of the invention were investigated for their potential for monitoring fermentation processes in the food industry. Fermentation of flour dough by yeast was used as a model food fermentation process.

In the preliminary experiment, ˜20 gm plain wheat flour (Pam's Brand) was mixed with 5, 25 and 50 ml of yeast solution in a 100 ml beaker. Yeast solution was prepared by mixing 1.5 g yeast powder (Edmond's Active Yeast) with 200 ml of 0.9% NaCl solution and allowing approximately 10 minutes for the yeast to swell before use.

For controls we used (a) 0.9% NaOH solution or (b) flour containing 50 ml yeast solution as prepared above but where the yeast solution had been autoclaved to kill the yeast The beakers containing various treatments were placed in glass jars (1 litre capacity; sealed air-tight) and incubated at 37° C. with sensors T-045, T-088 and T-140 made using laminate A1. At various times, the sensor colour and CO₂ levels were measured.

The colour change and CO₂% after 6 hours are shown in Table 5 below. Colour values are based on the colour wheel as described in Example 4. There was no CO₂ accumulation in either of the control experiments.

TABLE 5

Sensor colours

| Yeast (ml) | CO₂% | T-045 | T-088 | T0-140 |
|---|---|---|---|---|
| 5.0 | 1.0 | 17 | 18 | 18 |
| 25.0 | 15.0 | 12 | 18 | 18 |
| 50.0 | 24.0 | 2 | 12 | 18 |

FIG. 7 shows the colour change in the three sensors and the CO₂% over 20 hours for the 25 ml yeast suspension.

FIG. 8 shows the colour change of three sensors T-045, T-055 and T-88 when placed in a sealed container with 200 g flour mixed with 100 ml yeast solution. The sensors were made according the process of Example 1.

### Example 7

Sensors of the invention were used to measure CO₂ loss from the soil. The sensors were made according to the protocol in Example 1. The sensitivities were T-045, T-087.5 and T-140. The laminate used was type A 1.

A soil sample was obtained from a paddock at Ruakura Research Centre, Hamilton, New Zealand. All the plant materials consisting of roots and dead leaves were removed.

Two approximately 30.0 g portions of soil were obtained from this processed sample and were placed in two separate 100 ml beakers. One of these samples (control) was autoclaved to destroy all biological organisms.

The control and test beaker of soil were each placed into a separate glass jar (Agee jar). Two of each type of sensor was placed in each jar before tightly sealing the jar. CO₂ levels and colour changes were monitored as shown in FIG. 9. There was no CO₂ loss from the autoclaved (control) samples. In the test beaker, the non-autoclaved soil released CO₂ gradually.

The sensors changed colour from red to yellow in response to the CO₂ released from the soil in the test beaker. The most sensitive sensor, T-045 changed colour first, followed by T-088 then T-140. The colour changes after 48, 96 and 144 hours are shown in FIG. 10. The sensors labelled A are T-045 sensors, B are T-088 and C are T-140.

### Example 8

Sensor devices of the invention were made using four different acid-base indicator compositions. The sensors were made following the protocol outlined in Example 1 but Alizarin yellow R was substituted with Alizarin CI58000, Alizarin yellow GG or Thiazol yellow G (Titan yellow) to make four different sensor devices of the invention. Because of limited solubility the amount of Alizarin CI58000 was reduced to 150 mg per 100 ml glycerol-ethanol mixture. We note Alizarin CI58000 is incorrectly designated as Alizarin CI5800 in FIG. 11.

For each of the acid-base indicators, sensors of sensitivity T-045, T-88 and T-140 were made. The laminate used in each case was type A1.

To investigate the irreversibility of the sensors of the invention the colour changes of the sensors upon exposure to 1.0% CO₂ atmosphere were measured over 187 hrs, then for 25 hours after exposure to 0% CO, atmosphere (air).

FIG. 11 shows the results for the T-045 sensors made using various acid-base indicators. Three examples of each sensors type were used. One set (control) was placed in a closed plastic container in air atmosphere. The second set was placed in 1.0% CO₂ atmosphere in a large fish tank (capacity=24.3 litres). Colour changes in control and CO₂ groups were monitored over 187 hours At the end of 187 hours, the lid of the fish tank was opened to air atmosphere. The sensor colours were again evaluated after 25 hours.

The rate of colour change was slow for all the acid-base indicators in 1% CO₂ atmosphere. Colour change was not observed for any sensors stored in an air atmosphere (control).

The Alizarin yellow R colours (quantitated using the 18 point colour wheel) showed different patterns for different sensitivities. The changes in colours of other dyes were qualitatively described. Colour changes of varying degrees were also noted for other acid-base indicators.

The T-088 and T-140 sensors showed patterns parallel to that shown by the T-045 sensors except that the extent of colour change was less as was expected due to the differences in their sensitivities. These results are not shown.

After exposure to 0% CO₂ none of the sensors of the invention reverted to their original colour. All remained the colour that they were after maximal exposure to CO₂.

### Example 9

Sensor devices of the invention were made using different laminates. Sensors with sensitivity T-045, T-055, T-88, T-100 and T-140 were made according to the protocol in Example 1. Sensors of each sensitivity were made using three types of laminate film—type A1, type A2 and type A3, as described in Example 4.

The sensor devices were incubated in sealed plastic jars containing various levels of CO₂ from 0% (air) to 10.0%. The rate of colour change was recorded for each sensor at various CO₂ concentrations for 72 hours.

The results are shown in Table 6 below. Table 6 shows the time (hrs) required for the sensors to change colour from 18.0 to 1.0 on the colour wheel scale.

TABLE 6

| | T-045 | | | T-055 | | | T-088 | | | T-100 | | | T-140 | | |
|---|---|---|---|---|---|---|---|---|---|---|---|---|---|---|---|
| CO₂ % | A1 | A2 | A3 | A1 | A2 | A3 | A1 | A2 | A3 | A1 | A2 | A3 | A1 | A2 | A3 |
| 10 | 11 | 6 | 4 | 23 | 6 | 4 | 23 | 6 | 6 | 23 | 23 | 4-6 | 47 | 23 | 11 |
| 7.5 | 23 | 6 | 4 | 23 | 11 | 4 | 23 | 23 | 6 | 28.5 | 23 | 4-6 | 47 | 23 | 11 |
| 5 | 23 | 11 | 4 | 23 | 23 | 4 | 47 | 23 | 11 | 47 | 23 | 11 | >47 | 23-28.5 | 11 |
| 2 | * | 47 | 11 | * | 71.5 | 23 | * | * | 23 | * | * | 23 | * | * | 71.5 |
| 1 | * | * | 23 | * | * | 23 | * | * | 47-71.5 | * | * | 47-71.5 | * | * | * |

* did not change to colour 1 by 72 hours.
The results are based on duplicate measurements No variation between duplicates except for 1% CO₂; T-087.5; L3.

In some sensors colour changes were observed as early as 4 hours for one laminate at 2% CO₂ and for all laminates at 5% CO₂ and above. Typical examples of the effect of the laminate films for two different CO₂ concentrations are given are shown in FIG. 12. This Figure shows the colour changes of the sensors after 4 hrs in 2% CO₂ and 10% CO₂.

The T-045 sensors show a greater colour change in both 2% CO₂ and 10% CO₂ irrespective of the type of laminate film used. As the sensitivity of the sensor decreases, the colour change lessens, with T-140 barely changing in 2% CO₂ after 4 hrs.

The sensor devices of all sensitivities change colour less quickly when a laminate of A1 is used. Sensor devices made using laminate type A3 provided the quickest colour change.

FIG. 13 shows the colour profiles of the sensors made using laminate type A1 after exposure to various levels of CO₂ for 28 hrs. Clearly the sensor devices change colour more when exposed to a higher CO₂ concentration. The order of sensitivity decreases from the T-045 sensors to the T-140 sensors.

The results show that the colour change of the sensors of the invention is influenced both by the sensitivity of the acid-base indicator composition (the concentration of alkaline) and by the type of laminate film used. The effect of the laminate is independent of both the CO₂ concentration and the sensitivity of the acid-base indicator composition.

### Example 10

Unripe kiwifruit cv. Hayward that had been stored for up to 8 weeks were purchased from a coolstore/packhouse in Te Puke, New Zealand These kiwifruit were conditioned with 100 ppm ethylene for 24 hours and were placed in flow wrap packs (5 kiwifruit per pack) and left for ripening at 18-20° C. The perforation in the flow wrap was modified to provide sufficient ventilation so that the CO₂ concentration would be appropriate.

The flow packs contained about 110 perforations per pack, of 100 μm diameter micro-perforations. A previous study had established that these modifications would allow accumulation of 0.7% CO₂.

Sensors of the invention made according to the protocol in Example 1 were glued to a 250 um PVC backer and included in the packs when sealed. Sensors T-055, T-85 and T-135 were used.

The firmness of the kiwifruit was measured at four different points using a Sinclair Internal Quality device (SIQ-FT, Sinclair Systems International, LLC, Fresno, Calif., USA) before and after the experiment. Sinclair Internal Quality (IQ) devices are non-destructive impact sensors used to measure firmness of foods such as fruit. They are widely used in the food industry to estimate the ripeness of foods that get less firm as they ripen such as kiwifruit, avocados, stonefruit and the like.

The packaged kiwifruit were left at room temperature for 168 hrs. During the incubation the colour chance of the sensors and the accumulated CO₂% were monitored. The results of a typical experiment are shown in FIG. 14. Photographs of the packaged kiwifruit are provided in FIG. 15. In the photographs the top two sensors are T-055 (labelled A), with the middle two sensors T-085 (labelled B) and the bottom two sensors T-135 (labelled C).

### Results:

The average initial firmness of kiwifruit packed into flow wraps was 28 Sinclair IQ. After about 7 days ripening (=168 hrs), the average firmness decreased to 17 Sinclair IQ.

The CO₂% in the flow packs initially increased and then leveled off due to the ventilation. Sensors also changed their colours according to their sensitivity as can be seen in FIGS. 14 and 15.

### INDUSTRIAL APPLICABILITY

The CO₂ sensor devices of the invention provide a convenient means for the consumer to assess the ripeness of fruit. Once the device indicates the desired ripeness, the fruit can be consumed or refrigerated to slow the ripening process. Consumers can fruit produce confident that they will recognise the moment when it is at its best.

The CO₂ sensor devices of the invention also detect food spoilage by detecting the CO₂ emitted by food spoilage micro-organisms. The CO₂ sensor devices of the invention can also be used to indicate fermentation of food by detecting the CO, emitted by food fermentation micro-organisms.

1. A device for detecting CO₂ comprising:
(a) a support base;
(b) an acid-base indicator composition of pH about 11.5 to about 13.5 that stably changes colour upon exposure to CO₂; and
(c) a viewing cover;
wherein at least one of (a) and (c) is CO₂ permeable and wherein (b) includes at least one acid-base indicator that has a pKₗₙ of or above about 10.6 to about 12 and (b) is disposed between (a) and (c) such that the colour change of (b) can be observed through (c).

2.-14. (canceled)

15. A device according to claim 1 wherein the acid-base indicator is selected from the group comprising Alizarin yellow R, Alizarin yellow GG, Thiazole yellow G, and mixtures thereof.

16. A device according to claim 1 wherein the acid-base indicator composition has a pH of about 12 to about 13 and the acid-base indicator composition includes at least one acid-base indicator that has a pKₗₙ of about 11 to about 12.

17. A device according to claim 1 wherein the acid-base indicator composition comprises at least one buffer with at least one pKₐ of about 10 or above.

18. A device according to claim 1 wherein the concentration of acid-base indicator is 2 mM to 6 mM.

19. A device according to claim 1 wherein the device stably changes colour upon exposure to about 0.5% to about 15% CO₂.

20. A device according to claim 19 wherein the device stably changes colour upon exposure to about 0.5% to about 10% CO₂.

21. A device according to claim 19 wherein the device stably changes colour upon exposure to about 0.5% to about 5%.

22. A device according to claim 1 wherein the acid-base indicator composition comprises 5 mM Alizarin yellow R.

23. A device according to claim 1 for detecting CO₂ emitted by fruit, food micro-organisms, soil and micro-organisms.

24. A device according to claim 1 for detecting CO₂ emitted by climacteric fruit.

25. A method for detecting CO₂ from a CO₂ emitting source comprising:
(1) placing on or near the CO₂ emitting source, a device for detecting CO₂, wherein the device comprises
(a) a support base;
(b) an acid-base indicator composition of pH about 11.5 to about 13.5 that stably changes colour upon exposure to CO₂; and
(c) a viewing cover;
wherein at least one of (a) and (c) is CO₂ permeable and wherein (b) includes at least one acid-base indicator that has a pKₗₙ of about 10.6 to about 12 and (b) is disposed between (a) and (c) such that the colour change of (b) can be observed through (c); and
(2) detecting a colour change in (b).

26. A method according to claim 25 wherein the CO₂ emitting source is selected from the group comprising fruit, food microorganisms, soil and soil microorganisms.

27. A method for indicating fruit ripeness comprising
(1) placing on or near the fruit, a device for detecting CO₂, wherein the device comprises
(a) a support base;
(b) an acid-base indicator composition of pH about 11.5 to about 13.5 that stably changes colour upon exposure to CO₂; and
(c) a viewing cover;
wherein at least one of (a) and (c) is CO₂ permeable and wherein (b) includes at least one acid-base indicator that has a pKₗₙ, of about 10.6 to about 12 and (b) is disposed between (a) and (c) such that the colour change of (b) can be observed through (c); and
(2) detecting a colour change in (b).

28. A method according to claim 27 wherein the fruit is avocado, banana, pear or kiwifruit.

29. A method according to claim 25 wherein the device is placed on or up to about 10 cm from the fruit.

30. A method according to claim 25 wherein the device is placed inside packaging also containing the fruit.

31. A method according to claim 30 wherein the packaging is a clamshell packaging.
